(19) United States
(12) Patent Application Publication
(10) Pub. No.: US 2023/0323484 A1
(43) Pub. Date: Oct. 12, 2023

(54) METHOD FOR PREPARING TEST SOLUTION FOR PATHOGEN DETECTION PURPOSE,SYSTEM, KIT, DETECTION PRIMER AND METHOD THEREBY

(71) Applicant: TONGLI BIOMEDICAL CO., LTD, Suzhou (CN)

(72) Inventors: M QIAN, Suzhou (CN); Tongxin LI, Suzhou (CN); Mingxin QIAN, Suzhou (CN)

(21) Appl. No.: 18/023,793

(22) PCT Filed: Aug. 27, 2021
(86) PCT No.: PCT/CN2021/115118

§ 371 Date: Feb. 28, 2023

(30) Foreign Application Priority Data
Aug. 28, 2020 (CN) 202010889216.3

(51) Int. Cl. C12Q 1/70; C12Q 1/6851; G01N 1/34

(52) U.S. Cl. CPC C12Q 1/701; C12Q 1/6851; G01N 1/34; C12Q 1/6806

(57) ABSTRACT

A method for preparing test solution for pathogen detection purpose, system, kit, detection primer and method are provided. The method of preparing a test solution includes lysing the sample to be tested with a lysis buffer to release the nucleic acids contained in the sample to obtain a lysis buffer containing nucleic acids and/or pathogen nucleic acids; extracting the lysis buffer containing nucleic acids through a nucleic acid extraction device to obtain an extract containing host nucleic acids and/or pathogen nucleic acids; preparing the test solution for pathogen detection purposes from the extract. A large volume of samples can be used in the present application and it greatly improves sensitivity and specificity of the assay.

### CROSS-REFERENCE TO RELATED APPLICATIONS

This application claims priority to PCT Application No. PCT/CN2021/115118, having a filing date of Aug. 27, 2021, which claims priority to CN Application No. 202010889216.3, having a filing date of Aug. 28, 2020, the entire contents both of which are hereby incorporated by reference.

### SEQUENCE LISTING

This application includes a separate sequence listing in compliance with the requirement of 37 C.F.R.§.§ 1.824(a)(2)-1.824 (a)(6) and 1.824 (b), submitted under the file name “0062US01_SEQIDComplete”, created on Feb. 28, 2023, having a file size of 21.4 KB, the contents of which are hereby incorporated by reference.

### FIELD OF TECHNOLOGY

The following relates to method for preparing test solution for pathogen detection purpose, system, kit, detection primer and method thereby.

### BACKGROUND

The COVID-19 (SARS-CoV-2) is a single-stranded positive-sense RNA virus wrapped in protein, which invades the body mainly through the upper respiratory tract and digestive tract. The spike protein on the surface of the virus binds to the ACE2 receptor expressed by upper respiratory tract cells and digestive tract cells with high affinity and specificity, thereby entering the host cell, and using the host organelle for virus protein synthesis and virus replication. At present, there are two main clinical detection methods for the COVID-19: nucleic acid detection and immunological detection using antigens and antibodies.

For nucleic acid detection, such as blood, sputum, saliva, pleural effusion, chest/ascites, cerebrospinal fluid, bronchial/pulmonary lavage fluid, secretions (such as nasopharyngeal secretions), excrement (such as urine, feces), etc. are usually used as a liquid specimen for the extraction of viral nucleic acid; and reverse transcription quantitative polymerase chain reaction is currently the most widely used. RT-qPCR detection involves the following steps: 1) specimen collection and stabilization of viral RNA; 2) RNA extraction; 3) reverse transcription reaction or direct (one-step) RT-qPCR.

However, the current positive rate of COVID-19 detection by this method is only 30-50% as a result of limited RNA extraction technology. Due to the high affinity of COVID-19 with the ACE2 receptor expressed in the upper respiratory tract, nasal secretions collected by nasopharyngeal swabs, pharynx and tonsil secretions collected by oropharyngeal swabs, alveolar lavage fluid, etc. are often used as specimen. Due to the technological bottleneck of existing commercial extraction devices, the volume of existing specimens generally does not exceed 200-300 μL, resulting in low RNA yield. Commercial extraction devices include but are not limited to Qiagen's QIAamp Viral RNA Mini Kit and QIAamp DNA Blood Mini Kit, the nucleic acid extraction or purification kit (magnetic bead method) produced by Daan Gene Co., Ltd. of Sun Yat-sen University (Yue Sui Xie No. 20170583 and Yue Sui Xie No. 20150302), Viral RNA extraction kit (DP315-R) produced by Tiangen Biochemical Technology (Beijing) Co., Ltd.

In addition, the current laboratory testing process exposes medical staff to a cross-infected environment, and the current COVID-19 testing is still limited by specimen transportation, reagent materials and instruments, costs, laboratory technology, environment, and completion time.

It is currently known that once infected with COVID-19, as the host's immune cells respond, it will cause the host's body to produce different cytokines and other inflammatory proteins at different stages, which can cause a cytokine storm, and the result is a life-threatening inflammatory response, which typically leads to acute respiratory distress syndrome (ARDS) and organ failure and may even prevent the development of long-term immunity after the disease is cured. Therefore, targeted screening and dynamic detection of cytokine levels can help distinguish between the general infection and the patients who are more likely to suffer from severe COVID-19, so as to facilitate clinical management to improve the treatment effect and reduce the mortality rate. The current clinical methods for detecting cytokines are mostly from blood samples through immunological detection, such as ELISA, etc., to detect specific cytokine proteins through antibodies. However, these assays are time-consuming and cost, hard to meet clinical need for a rapid, dynamic and bedside monitoring to guide differential diagnosis, clinical staging and intervention including medication management and better prognosis.

### SUMMARY

An aspect relates to a method for preparing a test solution for pathogen detection using a sample to be tested. The pathogens include but are not limited to COVID 19.

The method includes the following steps:
1. lysing the sample to be tested with a lysis buffer to release the nucleic acids contained in the sample to obtain a lysis buffer containing nucleic acids, the nucleic acids include host nucleic acids, or when the sample to be tested contains pathogens, the nucleic acids also include pathogen nucleic acids;
2. extracting the lysis buffer containing nucleic acids through a nucleic acid extraction device to obtain an extract containing host nucleic acids, or when the sample to be tested contains pathogens, the extract also contains pathogen nucleic acids;
3. preparing the test solution for virus detection purposes from the extract;

The method also includes a step selected from at least one of the following:
A. controlling the environment where the extract is located to stabilize the nucleic acids in the extract so that the test solution contains the nucleic acids;
B. making the lysis buffer and/or washing buffer used in the extraction process contain tris(2-carboxyethyl)phosphine hydrochloride to accelerate and enhance the degradation of proteins or other substances and stabilize nucleic acids;
C. controlling the volume of the sample to be tested to 1-40 mL;
D. using a closed nucleic acid extraction device for the extraction.

A further aspect of the present disclosure is to provide a preparation system for preparing a test liquid. The test liquid is used for the detection of pathogens and is prepared from a sample to be tested. The pathogens include but are not limited to COVID 19, and the preparation system includes collection devices and extraction devices.

A further aspect of the present disclosure is to provide a kit for preparing a test liquid which is used for the detection of pathogens and is prepared from a sample to be tested. The pathogens include but are not limited to COVID 19. The kit includes a washing buffer; and/or, the kit includes a sample processing solution; and/or, the kit includes a deoxidizer; and/or, the kit includes one or more of lysis buffer, binding buffer, elution buffer and protease. Wherein, the lysis buffer and/or the washing buffer contains tris(2-carboxyethyl)phosphine hydrochloride.

A further aspect of the present disclosure is to provide an application of the above-mentioned preparation method and/or the above-mentioned preparation system and/or the above-mentioned kit in extracting pathogen nucleic acids, such as COVID 19 nucleic acids, and host nucleic acids in a sample to be tested.

A further aspect of the present disclosure is to provide primers for detecting the nucleic acids of the COVID 19 and the host nucleic acids. The primers include primer pairs for the nucleic acids of the COVID 19 and primer pairs for one or more of the host nucleic acids.

A further aspect of the present disclosure is to provide a nucleic acid detection method, which uses the aforementioned primers to perform quantitative PCR detection on a test solution separately or simultaneously, and the test solution is extracted by the aforementioned method, or the aforementioned system, or the aforementioned kit, and the test solution contains host nucleic acids; or the test solution also contains pathogen nucleic acids.

### BRIEF DESCRIPTION

Some of the embodiments will be described in detail, with references to the following Figures, wherein like designations denote like members, wherein:

FIG. 1 is a schematic structural diagram of a sample collector according to various embodiments of the present disclosure;

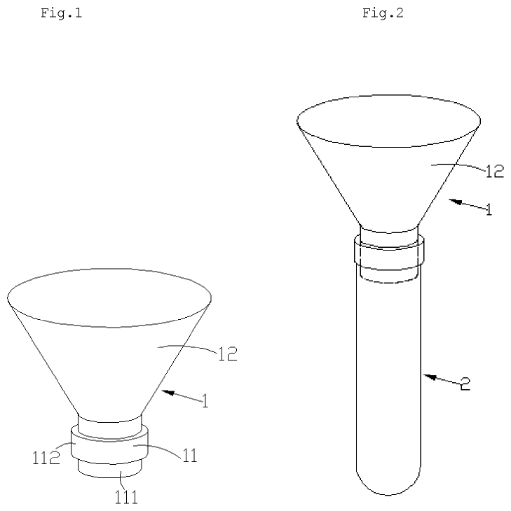

FIG. 2 is a schematic diagram of the structure when the sample collector and the sample storage container are connected according to various embodiments of the present disclosure;

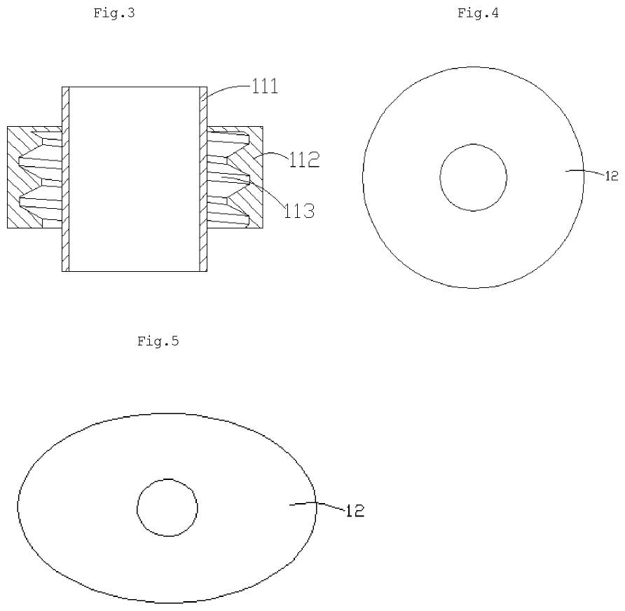

FIG. 3 is a cross-sectional view of the connection part of the sample collector according to various embodiments of the present disclosure;

FIG. 4 is a top view of a sample collector according to one embodiment; and

FIG. 5 is a top view of a sample collector according to another embodiment.

### DETAILED DESCRIPTION

For the purposes of promoting an understanding of the principles of the present disclosure, reference will now be made to embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the conventional art to which the disclosure relates.

### Definitions

Articles “a” and “an” are used herein to refer to one or more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result. The use herein of the terms “including”, “comprising”, or “having”, and variations thereof, is meant to encompass the elements listed thereafter and equivalents thereof as well as additional elements. As used herein, “and/or” refers to and encompasses any and all possible combinations of one or more of the associated listed items, as well as the lack of combinations were interpreted in the alternative (“or”).

Recitation of ranges of values herein are merely intended to serve as a shorthand method of referring individually to each separate value falling within the range, unless otherwise-indicated herein, and each separate value is incorporated into the specification as if it were individually recited herein. For example, if a concentration range is stated as 1% to 50%, it is intended that values such as 2% to 40%, 10%˜30%, or “1% to 3%”, etc., are expressly enumerated in this specification. These are only examples of what is specifically intended, and all possible combinations of numerical values between and including the lowest value and the highest value enumerated are to be considered to be expressly stated in this disclosure.

As used herein, “treatment”, “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

As used herein, the term “subject” and “patient” are used interchangeable herein and refer to both human and nonhuman animals. The term “nonhuman animals” of the disclosure includes all vertebrates, eg., mammals and non-mammals, such as nonhuman primates, sheep, dog, cat, horse, cow, chickens, amphibians, reptiles, and the like.

Unless otherwise defined, all technical terms used herein have the same meaning as commonly understood by one of ordinary skilled in the conventional art to which this disclosure belongs.

A Preparation System for Preparing a Test Liquid

Embodiments include a system capable of concentrating and extracting nucleic acids in large-volume samples, which can separate or remove genomic DNAs and retain pathogen nucleic acids and host nucleic acids. The system includes a collection device and an extraction device described in detail below.

The collection device (seen from FIG. 1 to 5)

The collection device includes a sample collector 1 and a sample storage container 2 detachably interconnected with the sample collector 1.

In some embodiments, the sample collector 1 includes a connecting part 11 and a collecting part 12. The connecting part 11 is detachably connected with the sample storage container 2. The collection part 12 and the connection part 11 are fixedly connected with each other or integrally formed. The collection part 12 has an opening, through which the liquid specimen enters the collection part 12 and finally enters the sample storage container 2. The cross-sectional area of the opening of the collecting part 12 is larger than the cross-sectional area of the connecting position where the collecting part 12 is connected.

In some embodiments, the cross section of the opening of the collecting part 12 is circular (seen from FIG. 4) or oval (seen from FIG. 5) to facilitate matching with body parts.

In some embodiments, the shape of the collecting part 12 includes but is not limited to an inverted frustum of a cone shape, and can also be any other feasible shape.

In some embodiments, the connecting part 11 includes a first part 111 having an internal passage communicating with the collecting part 12, and a second part 112 fixedly arranged outside the first part 111. The first part 111 is fixedly connected with the lower part of the collecting part 12 or the first part 111 is integrally formed with the lower part of the collecting part 12. An accommodating space for inserting the sample storage container 2 is formed between the inner wall of the second part 112 and the outer wall of the first part 111.

In at least one embodiment, an internal thread 113 is formed on the inner wall of the second part 112, an external thread is formed on the outer wall of the sample storage container 2. When the sample collector 1 is connected to the sample storage container 2, the sample storage container 2 is threadedly connected with the second part 112.

In some further embodiments, a gap is formed between the inner wall of the sample storage container 2 and the outer wall of the first part 111 when the sample collector 1 is connected to the sample storage container 2 (seen from FIG. 2), which helps to avoid contamination of the sample storage container 2 and contents therein, and also the sample collector 1.

In some embodiments, the detachable connection between the sample collector 1 and the sample storage container 2 includes, but is not limited to, snap connection, threaded connection, etc. In at least one embodiment, the sample collector 1 and the sample storage container 2 are connected by threaded mating.

In some embodiments, the sample storage container 2 may be a centrifuge tube or other kinds of container capable of containing liquid, such as a 50 mL centrifuge tube (29 cm*117 cm). When in use, one can fix the connecting part 11 of the sample collector 1 with the sample storage container 2, and then collect the sample like saliva, liquid containing saliva after rinsing with water, urine, etc. to be tested, after which unscrew the sample collector 1 and close the sample storage container 2 using a lid.

In some embodiments, the sample storage container 2 is added or pre-stored with sample processing substances used to inactivate, preserve, digest or release the nucleic acids in the sample.

The sample processing substances may be in any form, including but not limited to liquid state or solid state. When the sample processing substances in a solid state include but are not limited to a dry powder state, which further include but are not limited to freeze-dried powder.

In some further embodiments, the sample processing substances in a dry powder state quickly dissolve once they come into contact with the sample, producing the same or similar effect as the sample processing substances in a liquid state.

The sample processing substances may be a sample lysis solution, a sample preservation solution or a sample inactivation solution obtained by purchase or already disclosed. In an embodiment, it may be a sample processing substances containing triton (Triton X-100), tris(2-carboxyethyl)phosphine hydrochloride and Tris-HCl buffer in the composition, where the concentration of tris(2-carboxyethyl)phosphine hydrochloride may be 1˜20 mM.

The collection device can be used to achieve the collection of samples by the personnel to be examined and avoid cross-contamination during sample collection.

In some embodiments, the sample to be tested includes but is not limited to blood, body fluids, secretions, and excrement. In at least one embodiment, the sample to be tested is one or more of saliva, urine, nasopharyngeal swab, oropharyngeal swab, bronchial/lung lavage, cerebrospinal fluid, lymphatic fluid, ascites, amniotic fluid, peritoneal dialysis fluid, among which saliva samples may be preferred in embodiments, since saliva or liquid containing saliva after rinsing with water even in large volume e.g. 1-40 ml, can be easily collected, and also, their collection may be done by a subject himself/herself, which can avoid cross-infection between medical staffs and subjects. It had been proved through experiments that the detection of pathogen nucleic acids is achieved by collecting 1-2 ml of saliva from the subject infected with pathogens, extracting and testing according to the method of the present disclosure. Moreover, the sensitivity of the present disclosure is 16 times that of the existing mainstream nucleic acid extraction and detection commercial kits.

It should be noted that the nucleic acids in the sample to be tested come from a host and a single or multiple pathogens. The pathogens include but are not limited to viruses, bacteria, fungi or parasites. Parasites include, but are not limited to, various schistosomes, liver and lung flukes, tapeworms that lead to echinococcosis and neurocysticercosis as well as intestinal worms that lead to soil-transmitted helminth infections. The host includes humans and other mammals.

Cytokine is the general term for a variety of small molecular proteins secreted by cells and used for intercellular signal transduction, such as interleukin (IL), interferon (IFN), chemokine, colony stimulating factor (CSF) and tumor necrosis factor (TNF), etc.

In some embodiments, nucleic acids from a host include, but are not limited to, nucleic acids of one or more of cytokines, chemokines, and biomarkers. The cytokines, chemokines or biomarkers are cytokines, chemokines or biomarkers produced by the host after the pathogens enter the host.

In some further embodiments, the cytokines include but are not limited to one or more of IL1B, IL1RA, IL2, IL3, IL4, IL5, IL6, IL7, IL8, IL9, IL10, IL12p70, IL13, IL15, IL17A, IL23, IL25, IL27 and IL33, the chemokines include but are not limited to one or more of chemokines CCL1, CCL2, CCL3, CCL11, CXCL1, CXCL2, CXCL8, CXCL9, CXCL10 and CXCL11 and eosinophil-activated chemokine, the biomarkers include but are not limited to one or more of basic FGF2, CSF, GCSF, GMCSF, IFN, IFN γ, IP-10, MCP1, MIP1A, MIP1B, PDGFB, RANTES, TNF, TGFβ, TSLP, VEGFA, HO1, CRP, PCT, SAA, vWF, SELP and THBD. Or the cytokines include but are not limited to one or two or three or four or five of IL2, IL6, IL10, IL17A and IL13, or the biomarkers include but are not limited to one or two or three or four or more than four of HO1, CRP, IP-10, SAA, TNF, MCP1, IFN γ, vWF, SELP and THBD. Further the biomarkers at least include HO1.

COVID-19 infection is roughly divided into three stages, namely early lymphocyte decline, middle pneumonia, and late cytokine storm (CRS). COVID-19 infection will cause a series of serious symptoms, multiple organs of the body will be infected, and a number of cytokines will rise, and the so-called cytokine storm will appear.

The cytokines produced by the organism after invasion by COVID-19 include but are not limited to one or more of IL2, IL6 and IL10, the biomarkers produced by the organism after invasion by COVID-19 include but are not limited to one or more of HO1, CRP and IP-10, and SAA.

In some embodiments, nucleic acids from a host are mRNA.

In some embodiments, the volume of the sample to be tested is 1-40 mL. Such a large volume sample may be a mixture of saliva and liquid obtained by washing the mouth/throat using pure water or physiological saline, or other large-volume body fluids such as urine, bronchial/pulmonary lavage fluid, cerebrospinal fluid, lymphatic fluid, ascites, and amniotic fluid as well as peritoneal dialysate.

The extraction device
the extraction device comprises:
a receptacle defining an internal volume;
a removable cap for the receptacle, the cap having an internal side facing the internal volume of the receptacle and an external side facing away from the internal volume, the cap comprising a breather port communicating between the internal side and the external side and a sample connection port communicating between the internal side and the external side, the sample connection port comprising a first interlocking component for releasably locking the sample connection port to a cooperating second interlocking component, the internal side of the cap comprising a connection interface in fluid communication with the sample connection port;
a filter column adapted to be removably attached to the connection interface of the receptacle cap, the filter column having an open first end, an open second end, and an internal passage therebetween containing a substrate for collecting the nucleic acid;
a shipping container having an open end and defining a volume adapted to contain the filter column, the shipping container adapted to releasably engage the filter column for detaching it from the connection interface of the receptacle cap, the shipping container further comprising a removable lid for temporarily sealing the filter column within the shipping container.

Further, the detailed structure of this extraction device (Manually Operated Extraction System, referred to as MOES) is shown in paragraphs 0035 to 0067 of the patent CN201480042043.4 named “System and Method for Collecting Nucleic Acid Samples”, the entire contents of which are hereby incorporated by reference, and shown in FIGS. 1 to 6. The structure involved in the patent and the reagents and methods used are incorporated into this application in full, as long as there is no conflict with the scheme of this application, they can be applied to the present application.

In some embodiments, oxygen scavengers or antioxidants are added or pre-stored in the shipping container to control the oxygen content in the shipping container to be less than 0.01-1%, to further protect and stabilize the extracted nucleic acids.

The term deoxidizers, also known as oxygen scavengers or oxygen absorbents, are additives that can absorb oxygen to slow down the oxidation of the protected object. They are a group of chemical mixtures that easily react with free oxygen or dissolved oxygen. The deoxidizers may remove the residual oxygen in the sealed environment to prevent the object to be protected from discoloration, deterioration, and oil rancidity due to oxidation, and also inhibit the growth of molds, aerobic bacteria and harmful organisms by packing them in a sealed bag (similar to a desiccant bag) with a certain degree of air permeability and strength.

The nucleic acid extraction step can be performed in a closed environment to avoid cross-contamination between samples, prevent samples from polluting the external environment, and effectively protect the safety of experiment operators by adopting the extraction device for nucleic acid extraction.

The collection device and extraction device of this embodiment enable the collection of large samples, such as sample collection volumes of 1 to 40 mL and allow effective concentration of large volume samples to 100 μL and below, such as 60 μL, etc.; thus, they can be used for pooled population-based epidemiological surveys which combined with multiple small volume samples (100 to 200 copies of nasopharyngeal swabs) from different people, saving cost for labor and testing materials.

A Preparation Method for Preparing the Test Solution

Embodiments include a preparation method for preparing the test solution.

The method includes the following steps:
1) collecting sample to be tested using the collection device described above;
2) lysing the sample to be tested with a lysis buffer to release the nucleic acids contained in the sample to obtain a lysis buffer containing nucleic acids, the nucleic acids include host nucleic acids, or when the sample to be tested contains pathogens, the nucleic acids also include pathogen nucleic acids;
3) extracting the lysis buffer containing nucleic acids through the nucleic acid extraction device described above to obtain an extract containing host nucleic acids, or when the sample to be tested contains pathogens, the extract also contains pathogen nucleic acids;
4) preparing the test solution for virus detection purposes from the extract.

The method also includes a step selected from at least one of the following:
A. Controlling the environment where the extract is located to stabilize the nucleic acids in the extract so that the test solution contains the nucleic acids;
B. making the lysis buffer and/or washing buffer used in the extraction process contain tris(2-carboxyethyl)phosphine hydrochloride to accelerate and enhance the degradation of proteins or other substances and stabilize nucleic acids;
C. controlling the volume of the sample to be tested to 1-40 mL;
D. using a sealed nucleic acid extraction device for the extraction.

In some embodiments, in the step A, the oxygen content in the environment where the extract is located is controlled to be less than 1%, for example, less than 0.9%, 0.8%, 0.7%, 0.6%, 0.5%, 0.4%, 0.3%, 0.2%, 0.1% etc. For example, the oxygen content in the environment is about 0.01%˜1%, for example, about 0.01%˜0.1%.

In some further embodiments, the oxygen content can be controlled by adding oxygen scavengers to the environment, such as add oxygen scavengers to the environment where the extract is located after the extraction is completed. Among them, the environment includes the environment before the start of extraction, during the extraction process, or after the extraction.

In some embodiments, the oxygen content in the environment after the extraction is mainly controlled to further stabilize and protect the nucleic acid by keeping the extract in the environment with low oxygen content.

In some embodiments, the extraction step includes passing the lysis buffer containing nucleic acids through a filter column for adsorption, and then passing a washing buffer for washing, and then eluting the filter column with an eluent to obtain a test solution, where at least the adsorption and washing are performed in a closed environment. Among them, the nucleic acid adsorption step can be to use a filter column treated with a binding buffer for adsorption, or to mix a lysate containing nucleic acids with a binding buffer and then pass through the filter column for adsorption.

Further, the washing buffer includes absolute ethanol.

The term tris-(2-carboxyethyl)-phosphine hydrochloride (TCEP-HCl): also known as TCEP hydrochloride, tris(2-carboxy) phosphine hydrochloride, tris(2-Carboxyethyl) phosphine hydrochloride. TCEP-HCl is a pure, odorless and stable tris(2-carboxyethyl)phosphine crystal, which is a sulfhydryl-free compound that can efficiently reduce the disulfide bonds of proteins and polypeptides. TCEP-HCl is stable at room temperature, resistant to air oxidation, stable in water-soluble buffer, acid and lye, and can inhibit RNase enzyme activity. Dissolving tris(2-carboxyethyl)phosphine hydrochloride in absolute ethanol can further protect and stabilize the extracted nucleic acids.

In some embodiments, the concentration of the tris(2-carboxyethyl)phosphine hydrochloride in the lysis buffer or the washing buffer is 1-20 mM.

In some embodiments, the extraction method of the present disclosure can further isolate or remove genomic DNA, retain viral RNA and host RNA, and use different solid-liquid phase combinations to specifically extract the substances to be analyzed or specifically exclude interfering substances.

In some embodiments, the method of the present disclosure can extract a large-volume sample to be tested, which significantly improves the detection sensitivity, and the obtained nucleic acids also include short fragment nucleic acids, that is, free nucleic acids. Furthermore, the RNA and DNA extracted by the method of the present disclosure can remain stable for a long time at room temperature. Therefore, the method of the present disclosure solves the capacity limitation of the existing commercial extraction device for extracting liquid, and is suitable for operation in various laboratory environments such as high-end and low-end, and has no special requirements on the laboratory environment and technical conditions.

Primers and Method for Detecting Nucleic Acids to be Tested

Embodiments include a detection method capable of simultaneously detecting nucleic acids of different pathogens and host nucleic acids in the same sample. This is beneficial to monitor the dynamic changes of the body's immune response to pathogens, such as the dynamic changes of cytokines, which is beneficial to the early differential diagnosis of infectious diseases, and is beneficial to guide clinical intervention and medication. It has obviously practical value for better clinical staging, management and prognosis.

In some embodiments, the primers include primer pairs for the nucleic acids of the COVID 19 and primer pairs for one or more of the host nucleic acids.

In some further embodiments, the primers further comprise primer pairs for housekeeping genes of host, the housekeeping genes of host are present in both a healthy host and an infected host, so that the primer pairs for the housekeeping genes of host can be used as an internal standard. The primer pairs shown in SEQ ID NO. 29˜30 and SEQ ID NO. 129˜136 are primer pairs designed for RNA polymerase 2, and SEQ ID NO. 127˜128 are primer pairs designed for Beta microglobulin-2.

In some further embodiments, the primer pairs are primer pairs that span introns. They can only amplify the cDNA transcribed from mRNA after the splicing is completed. The introns in the sequence have been cut out, genomic DNA (gDNA) will be not amplified, and the interference of gDNA is avoided.

The primer pairs for various nucleic acids in this application are selected from SEQ ID NO.1-144. Among all the primer pair sequences, SEQ ID NO: 1 and SEQ ID NO: 2 are the 1st primer pair, SEQ ID NO: 3 and SEQ ID NO: 4 are the 2nd pair, SEQ ID NO: 5 and EQID NO: 6 are the 3rd primer pair, SEQ ID NO: 7 and SEQ ID NO: 8 are the 4th primer pair, and so on. The odd number is the upstream primer, the even number is the downstream primer, and the primers are written in order from the 5′ end to the 3′ end.

### Example 1

The preparation of the test solution of this example and the materials and specific steps used for PCR detection are as follows:
1. Reagents
1a. Lysis Buffer (LB).
1b. Binding Buffer (BB).
1c. Washing Buffer (WB). Tris(2-carboxyethyl)phosphine hydrochloride is dissolved in absolute ethanol, and the concentration of tris(2-carboxyethyl)phosphine hydrochloride is 1-20 mM.
1d. Elution Buffer (EB).
1e. Proteinase K. −20° C. to save.
1f. Anhydrous ethanol (provided by yourself)
2. Devices and materials
2a. Lysis tube: 50 ml centrifuge tube
2b. Eluent collection tube: 1.5 ml microcentrifuge tube
2c. 30 ml disposable syringe (screw connector)
2d. 5-10 ml disposable syringe (provided by yourself)
2e. High-speed microcentrifuge (≥12000 G)
2f. 60° C. water bath or dry heat module
2g. Anhydrous ethanol
3. RT-qPCR detection

Quantitative nucleic acid PCR detection has one-step RT-qPCR or two-step RT-qPCR. The former is that RT reactions and PCR reactions are in the same test tube, which uses target primers for RT reactions to produce specific cDNA fragments, and then uses the same specific primers for PCR amplification reactions. The reaction process does not need to open the test tube lid, which greatly reduces pollution. The latter is that first uses random primers for RT reactions to convert the RNA in the extract into corresponding cDNA, and then adds one or more pairs of specific primers to specifically amplify the specific target cDNA (target nucleic acid) and carry out quantitative detection.

There are usually two methods for real-time quantitative monitoring of PCR products: 1. Use non-specific fluorescent dyes such as SYBR Green to chimerize into the double-stranded DNA fragments of PCR products to generate fluorescence. 2. Fluorescent molecules and quenching groups are labeled with nucleic acid sequence-specific probes that complement and hydrolyze specific PCR product sequences during the PCR reaction, releasing the quenching groups and resulting in fluorescence.

The following is an example of a two-step RT-qPCR, that is, RT reaction uses Moloney Murine Leukemia Virus Reverse Transcriptase (M-MLV RT, M1701, Promega, WI, USA), and then performs SYBR Green qPCR (QuantiNova SYBR Green RT-PCR Kit, Cat No. 208054, QIAGEN GmbH, Hilden, Germany). In practical applications, one-step RT-qPCR or 2-step RT-qPCR can often be selected according to specific needs, as are the fluorescent chimeric dye method and nucleic acid sequence-specific probe method. The following examples are not listed in specific probes.

4. Sample Source

Nasopharyngeal swabs from patients with inflammations and infections were collected and centrifuged for 10 min (3000 g), and 2-20 ml of supernatant was aspirated in a sample storage container pre-stored with sample processing solution and stored at −20° C. Pseudovirus of COVID-19 was added to the samples from patients who have recovered or cured from anti-inflammatory treatment for subsequent experiments.

5. Extraction of Free Nucleic Acid
A. Thaw the frozen samples at room temperature, centrifuge at high speed for 3 minutes (12000 g) and reserve the supernatant for later use.
B. Pipette LB 1.9 ml to 50 ml centrifuge tube (lysis tube), add 2 ml of sample after high-speed centrifugation, and mix thoroughly. Add 100 μl Proteinase K solution and mix well again.
C. Cover the pyrolysis tube and place it in a 60° C. water bath (or dry heat module at 60° C.) for 30 minutes. Remove the lysis tube and let it cool down at room temperature for 5-10 minutes.
D. Add 8 ml of BB, vortex for 30 seconds, and leave it quietly for 3-5 minutes.
E. After pulling out the plunger of the 30 ml syringe, tightly connect the syringe with the Luer locking sleeve of the extraction device and place it vertically. Transfer the lysate to the syringe, insert the plunger of the syringe, apply pressure slowly and evenly, and inject the lysate into the extraction device. Note that when the liquid flows out of the adsorption column, it should be intermittently dripping, and the entire time of pushing process should be controlled within 90 seconds to 120 seconds. Push in the remaining air to remove the remaining liquid.
F. Aspirate the WB 4 ml to 10 ml syringe, connect with the Luer locking sleeve, push the WB through the extraction device to clean the adsorption column. Push in the remaining air to remove the remaining liquid.
G. Aspirate 4 ml of anhydrous alcohol (Ethanol), connect with the Luer locking sleeve, and push in the absolute ethanol (Ethanol) through the extraction device to clean the adsorption column. Push in air to remove residual liquid.
H. Rotate counterclockwise to remove the adsorption column, close the adsorption column using a cap, place in a 1.5 ml microcentrifuge tube, centrifuge at high speed for 3 minutes (12000 g) to remove residual ethanol. Remove the cap of the adsorption column and leave it at room temperature for 5-10 minutes to allow the residual ethanol to evaporate.
I. Seal the adsorption column with nucleic acids and store it in the transport container; perform the following steps when needed.
J. Place the adsorption column in a clean 1.5 ml microcentrifuge tube, add 60 μl of elution buffer (EB) to the adsorption membrane in the adsorption column, close the adsorption column using a cap, and let it stand at room temperature for 5 minutes.
k. Centrifuge the centrifuge tube containing the adsorption column at high speed for 3 minutes (12000 g) and collect the eluate. The eluate containing the separated free nucleic acids may be used as a template for PCR, qPCR or other molecular detection, or frozen and stored for later use.

5. Removal of DNA Secondary Structure (DNA Secondary Structure in the Template Needs to be Removed as it has an Effect on PCR Amplification)

0.5 μg of random primers (synthesized by Shanghai Biotech, 6 random pr (5′-NNN NNN-3′); 9 random pr (5′-NNN NNN NNN-3′)) and 15-20 μl of RNA template (eluate collected in the previous step) were mixed to a total volume of 20 μl at 4° C., and then PCR amplification was performed with an amplification procedure of 70° C. for 5 mins; the amplification products were used in the next experiments or stored at 4° C. for a short period (1-2d).

Reverse transcription (25 ul reaction system)

MMLV enzyme system: Promega M170, abbreviated MMLV.

Rnase inhibitor: Rnasin N2525, abbreviated as RNasin.

The reaction system is as follows.

| | | |
|---|---|---|
| M-MLV 5X Reaction Solution | 5 | μl |
| dNTP (25 mM) | 2 | μl |
| Rnasin (0.625 ul/25 ul) | 0.7 | μl |
| MMLV (200 U/25 ul) | 1 | μl |
| Product after secondary structure removal (i.e. random primer + | 12 | μl |
| RNA template) | | |
| RNase free H₂O | 4.3 | μl. |

PCR amplification, program: 42° C., 60 mins.

The products were stored at 4° C. for a short period (5-7d).

6. Quantitative PCR Detection of Free Nucleic Acid DNA (Using QIAGEN Fluorescent Quantitative PCR MIX)

Quantitative PCR detection is to amplify unique fragments, and at the same time chimerize into the amplified products through the fluorescent dye SYBR Green, to generate fluorescence signals with excitation wavelengths of 480 nm and radiation wavelengths of 520 nm, to achieve quantitative detection of amplified products, at the same time, specific amplification products were determined by melting curve analysis.
A. 2.0×qPCR reaction solution: vortex and shake at room temperature to fully thaw the reaction solution. Take an appropriate amount of the reaction solution, for example, for the total volume of 20 μl PCR, take 10 μl of the reaction solution, add 2.0 μl of primer pairs, mix well, then add to the PCR reaction tube.
B. Pipette 8.0 μl of the eluent (template) into the PCR reaction tube, close the tube using a cap, mix well, and centrifuge until the reaction solution collects at the bottom of the tube. Place the PCR reaction tube in the reaction module of the qPCR instrument.
C. PCR program setting:
(1). Excitation wavelength is 480 nm, radiation wavelength is 520 nm, or select the preset wavelength suitable for HAM or SYBR Green. The reaction volume is 20 μl.
(2). A PCR protocol run with BioRad CFX instrument
1 95.0° C. for 3:00
2 95.0° C. for 0:10
3 60.0° C. for 0:10
4 72.0° C. for 0:30
+Plate Read
5 GOTO 2, 44 more times
6 Melt Curve 65.0 to 95.0° C., increment 0.5° C., 0:05+Plate Read.
END

The primer pairs and the assay results are shown in Table 1.

TABLE 1

| | Primer | | Test 1 | | Test 2 | | Test 3 | |
|---|---|---|---|---|---|---|---|---|
| target | name | Primer sequence | Ct | Tm | Ct | Tm | Ct | Tm |
| N gene | NCPN3 | aactcaagccttaccgcaga | 33.3 | 75.5 | 32.67 | 76 | 33.57 | 75.5 |
| | | (SEQ ID NO. 117) | | | | | | |
| | | Tgcagcaggaagaagagtca | | | | | | |
| | | (SEQ ID NO. 118) | | | | | | |
| BETA | M11 | cgcgctactctctctttctgg | 25.35 | 79 | 24.76 | 79 | 25.1 | 79 |
| MICROGLOBULIN- | | (SEQ ID NO. 127) | | | | | | |
| 2 | | Agtcaacttcaatgtcggatgg | | | | | | |
| | | (SEQ ID NO. 128) | | | | | | |
| IL6 | B9 | tacatcctcgacggcatctca | 29.03 | 77.5, | 26.38 | 78 | 26.67 | 78 |
| | | (SEQ ID NO. 25) | | 81.5 | | | | |
| | | gcctctttgctgctttcacac | | | | | | |
| | | (SEQ ID NO. 26) | | | | | | |
| N gene | NCPN4 | ttcggaagagacaggtacgtt | 40.2 | 75 | N/A | None | 38.42 | 76 |
| | | (SEQ ID NO. 27) | | | | | | |
| | | cacacaatcgatgcgcagta | | | | | | |
| | | (SEQ ID NO. 28) | | | | | | |
| RNA | P5 | tgagggcactggaaattgtat | 25.41 | 78 | 25.24 | 78 | 25.12 | 78 |
| polymerase | | (SEQ ID NO. 29) | | | | | | |
| 2 | | ctattaatgtggcgattgaccga | | | | | | |
| | | (SEQ ID NO. 30) | | | | | | |
| N gene | NCPN5 | ggtcatgtgtggcggttcacta | 30.92 | 77 | 35.41 | 76 | 34.42 | 76.5 |
| | | (SEQ ID NO. 31) | | | | | | |
| | | gcataagcagttgtggcatctc | | | | | | |
| | | (SEQ ID NO. 32) | | | | | | |
| N gene | NCPN6 | caggcacaggtgttcttactga | 34.63 | 73 | 43.25 | None | 34.3 | 73 |
| | | (SEQ ID NO. 33) | | | | | | |
| | | tgccaaattgttggaaaggca | | | | | | |
| | | (SEQ ID NO. 34) | | | | | | |
| N gene | NCPN7 | caatgctgcaatcgtgctac | 36.39 | 75.5 | 33.64 | 78.5 | 33.78 | 75 |
| | | (SEQ ID NO. 35) | | | | | | |
| | | tgccgcctctgctcccttctg | | | | | | |
| | | (SEQ ID NO. 36) | | | | | | |

### Example 2

The method of this example is essentially the same as that of Example 1, with the following differences.
1. The different sample sources of this embodiment are shown in the following table 2. Pseudovirus of COVID-19 were added to all the samples.

TABLE 2

| | | | Quantity of |
|---|---|---|---|
| NO. | Specimens | Volume | Specimens |
| A | plasma/serum | 2 ml | 2 |
| B | plasma/serum | 2 ml | 2 |
| C | Mixing of multiple nasopharyngeal swabs | 2 ml | 2 |
| D | urine | 8 ml | 2 |

2. The differences in extraction methods are as follows.

Sample A was processed in the same way as in Example 1.

Sample B and sample C were extracted with the first binding buffer according to the same method as in Example 1 to obtain the first eluate for the assay (the eluate was recorded as the first column pass), and the liquid from the lysate column in Step E was collected and the second binding buffer was added to the liquid to extract the free nucleic acid according to the same method as in Example 1 (the eluate was recorded as the second column pass). Where the main component of the first sorbent is anhydrous ethanol, and the volume is 2 mL; the main component of the second sorbent is anhydrous ethanol and guanidine isothiocyanate, and the volume is 7 mL.

Sample D was treated in the same way as sample B or sample C, except that the volume of binding buffer added is 4 times the volume added in sample B or sample C.

The primer pairs in this example were shown in Table 3, and the results of the assay using each primer pair for each eluate of each of the above samples, respectively, were shown in Table 4.

TABLE 3

| target | Primer name | Primer sequence |
|---|---|---|
| N gene | NCPN3 | Aactcaagccttaccgcaga (SEQ ID NO. 117) |
| | | tgcagcaggaagaagagtca (SEQ ID NO. 118) |
| K11 | K11 | GCCTGCTGAAAATGACTGAAT (SED IQ NO. 143) |
| | | ATTAGCTGTATCGTCAAGGCAC (SED IQ NO. 144) |
| RNA | P5 | tgagggcactggaaattgtat (SEQ ID NO. 29) |
| polymerase | | ctattaatgtggcgattgaccga (SEQ ID NO. 30) |
| 2 | | |
| IL2 | C4 | ccaaactcaccaggatgctcac (SEQ ID NO. 43) |
| | | ccagaggtttgagttcttcttc (SEQ ID NO. 44) |
| IL2 | C5 | gacccagggacttaatcagca (SEQ ID NO. 45) |
| | | tgtctcatcagcatattcacaca (SEQ ID NO. 46) |
| IL6 | B7 | SED IQ NO. 21 |
| | | SED IQ NO. 22 |
| IL6 | B9 | tacatcctcgacggcatctca (SEQ ID NO. 25) |
| | | gcctctttgctgctttcacac (SEQ ID NO. 26) |
| N gene | NCPN2 | ctttgctgctgcttgacaga (SED IQ NO. 115) |
| | | gccttgttgttgttggcctt (SED IQ NO. 116) |
| RdRp | RdRp-1 | agatttggacctgcgagcg (SED IQ NO. 113) |
| | | gagcggctgtctccacaagt (SED IQ NO. 114) |

TABLE 4

| | | test1/Cq | | test2/Cq | |
|---|---|---|---|---|---|
| | | the first | the second | the first | the second |
| Primer | NO. of | column | column | column | column |
| name | sample | pass | pass | pass | pass |
| M11 | A1 | 22.02 | — | 21.46 | — |
| | A2 | 21.35 | — | 21.29 | — |
| | B1 | 27.44 | 28.09 | 27.7 | 27.94 |
| | B2 | 25.79 | 26.86 | 24.98 | 26.72 |
| | C1 | 21.32 | 18.56 | 22.06 | 18.38 |
| | C2 | 21.94 | 18.54 | 20.89 | 18.61 |
| | D1 | 24.22 | 18.06 | 23.93 | 18.11 |
| | D2 | 25.05 | 20.05 | 24.55 | 19.77 |
| K11 | A1 | 23.26 | — | — | — |
| | A2 | 23.08 | — | — | — |
| | B1 | 25.77 | 25.6 | — | — |
| | B2 | 22.39 | 26.47 | — | — |
| | C1 | 19.45 | 20.11 | — | — |
| | C2 | 19.64 | 20.55 | — | — |
| | D1 | 21.41 | 20.51 | — | — |
| | D2 | 21.29 | 19.67 | — | — |
| B11 | A1 | 25.13 | — | 25.27 | — |
| | A2 | 26.07 | — | 26.09 | — |
| | B1 | 29.15 | 30.54 | 29.24 | 31.36 |
| | B2 | 24.59 | 30.32 | 24.43 | 30.31 |
| | C1 | 21.59 | 22.85 | 21.52 | 23.18 |
| | C2 | 22.49 | 21.87 | 21.88 | 22.08 |
| | D1 | 26.61 | 24.03 | 26.53 | 23.67 |
| | D2 | 24.13 | 25.3 | 24 | 25.17 |
| NCPN3 | A1 | 29.97 | — | — | — |
| | A2 | 29.22 | — | — | — |
| | B1 | 30.13 | 30.42 | — | — |
| | B2 | 28.44 | 31.11 | — | — |
| | C1 | 25.49 | 24.47 | — | — |
| | C2 | 24.6 | 24.99 | — | — |
| | D1 | 27.63 | 25.9 | — | — |
| | D2 | 28.14 | 26.52 | — | — |
| C4 | A1 | 32.9 | — | 32.19 | — |
| | A2 | 30.54 | — | 30.44 | — |
| | B1 | 36.85 | 35.45 | 31.47 | 32.88 |
| | B2 | 31.25 | 34.07 | 32.04 | 33.07 |
| | C1 | 28.68 | 28.63 | 28.71 | 28.94 |
| | C2 | 28.32 | 29.09 | 27.69 | 28.37 |
| | D1 | 30.23 | 29.19 | 29.37 | 28.8 |
| | D2 | 32.06 | 30.3 | 31.04 | 30.69 |
| C5 | A1 | 31.23 | — | 28.53 | — |
| | A2 | 29.4 | — | 27.92 | — |
| | B1 | 31.59 | 31.63 | 29.08 | 28.63 |
| | B2 | 29.19 | 31.64 | 27.22 | 29.02 |
| | C1 | 28.09 | 28.66 | 25.26 | 26.18 |
| | C2 | 28.48 | 28.6 | 26.07 | 26.57 |
| | D1 | 27.09 | 28.59 | 26.57 | 26.21 |
| | D2 | 30.32 | 29.03 | 28.91 | 26.58 |
| B7 | A1 | 28.25 | — | 28.32 | — |
| | A2 | 27.61 | — | 27.46 | — |
| | B1 | 28.86 | 29.82 | 28.96 | 29.62 |
| | B2 | 26.95 | 30.07 | 27.15 | 29.07 |
| | C1 | 23.52 | 24.07 | 24.06 | 23.65 |
| | C2 | 23.53 | 23.52 | 23.18 | 23.58 |
| | D1 | 26 | 25.3 | 25.65 | 24.9 |
| | D2 | 27.72 | 26.02 | 27.67 | 25.81 |
| B9 | A1 | 29.19 | — | — | — |
| | A2 | 25.2 | — | — | — |
| | B1 | 26.41 | 27.45 | — | — |
| | B2 | 25.53 | 27.3 | — | — |
| | C1 | 22.53 | 25.61 | — | — |
| | C2 | 25.41 | 26.29 | — | — |
| | D1 | 24.28 | 25.07 | — | — |
| | D2 | 26.15 | 26.7 | — | — |
| NCPN2 | A1 | 37.81 | — | — | — |
| | A2 | 34.43 | — | — | — |
| | B1 | 44.4 | 34.78 | — | — |
| | B2 | 31.86 | 33.7 | — | — |
| | C1 | 29.05 | 30.52 | — | — |
| | C2 | 28.26 | 28.7 | — | — |
| | D1 | 38.13 | 31.47 | — | — |
| | D2 | 32.57 | 34.73 | — | — |
| RdRp-1 | A1 | 26.49 | — | — | — |
| | A2 | 27.24 | — | — | — |
| | B1 | 29.2 | 30.64 | — | — |
| | B2 | 25.6 | 32.21 | — | — |
| | C1 | 23.18 | 27.16 | — | — |
| | C2 | 24.4 | 25.43 | — | — |
| | D1 | 29.51 | 28.01 | — | — |
| | D2 | 26.54 | 28.65 | — | — |

As seen from Table 4, different extraction methods are feasible for different types of samples, and the amount of target fragments obtained can be controlled by changing the ratio of each component of the binding buffer to adapt to different testing requirements, and M11, K1, and P1 can be used as internal standard markers, and all the above primers are feasible.

### Example 3

In this example, samples from different patient sources than in Example 2 were used, and the extraction of free nucleic acids from the samples was performed according to the method of Example 2, and no pseudovirus of COVID-19 was added to Sample A and Sample B. The results of each primer and experiment were shown in Table 5.

TABLE 5

| | Primer name | | | | | |
|---|---|---|---|---|---|---|
| | M11 | | NCPN3 | | K11 | |
| | the | the | the | the | the | the |
| | first | second | first | second | first | second |
| Name of | column | column | column | column | column | column |
| sample | pass/Cq | pass/Cq | pass/Cq | pass/Cq | pass/Cq | pass/Cq |
| A1 | N/A | — | 40.09 | — | 24.01 | — |
| A2 | N/A | — | N/A | — | 24.72 | — |
| B1 | N/A | N/A | N/A | N/A | 28.22 | 31.37 |
| B2 | N/A | N/A | N/A | N/A | 23.13 | 28.29 |
| C1 | 39.16 | N/A | 37.23 | N/A | 19.7 | 22.46 |
| C2 | 42.03 | N/A | 38.06 | 44.47 | 20.72 | 22.28 |
| D1 | 39.81 | N/A | 37.54 | 33.9 | 25.46 | 24.25 |
| D2 | 37.27 | N/A | 36.66 | 35.23 | 22.25 | 25.29 |
| | Primer name | | | | | |
| | P5 | | C4 | | C5 | |
| | the | the | the | the | the | the |
| | first | second | first | second | first | second |
| Name of | column | column | column | column | column | column |
| sample | pass/Cq | pass/Cq | pass/Cq | pass/Cq | pass/Cq | pass/Cq |
| A1 | 24.61 | — | N/A | — | 45.66 | — |
| A2 | 25.58 | — | N/A | — | N/A | — |
| B1 | 28.75 | 29.9 | N/A | N/A | N/A | N/A |
| B2 | 24.04 | 28.85 | 45.55 | N/A | N/A | N/A |
| C1 | 20.6 | 23.13 | 36.03 | 42.71 | 41.09 | N/A |
| C2 | 21.75 | 23.14 | 38.75 | 39.87 | N/A | N/A |
| D1 | 26.4 | 25.09 | 45.44 | N/A | N/A | N/A |
| D2 | 23.28 | 25.43 | 37.03 | N/A | 38.47 | N/A |
| | Primer name | | | | | |
| | B7 | | B9 | | NCPN2 | |
| | the | the | the | the | the | the |
| | first | second | first | second | first | second |
| Name of | column | column | column | column | column | column |
| sample | pass/Cq | pass/Cq | pass/Cq | pass/Cq | pass/Cq | pass/Cq |
| A1 | N/A | — | 31.66 | — | 45.83 | — |
| A2 | 43.03 | — | 29.5 | — | N/A | — |
| B1 | N/A | N/A | N/A | N/A | N/A | N/A |
| B2 | 43.41 | N/A | 27.3 | 34.22 | 39.39 | N/A |
| C1 | 33.55 | 35.28 | 20.42 | 24.09 | 37.38 | 38.69 |
| C2 | 36.25 | 37.02 | 24.35 | 28.09 | 37.58 | 42.13 |
| D1 | 41.21 | 38.23 | 28.64 | 29.89 | 41.77 | 40.92 |
| D2 | 36.17 | 40.24 | 23.53 | 28.22 | 41.5 | N/A |

Primer name
RdRp-1

| Name of | the first | the second |
|---|---|---|
| sample | column pass/Cq | column pass/Cq |
| A1 | 25.31 | — |
| A2 | 26.03 | — |
| B1 | 28.38 | 31.12 |
| B2 | 23.61 | 29.19 |
| C1 | 21.27 | 24.27 |
| C2 | 22.2 | 23.39 |
| D1 | 27.5 | 25.44 |
| D2 | 23.7 | 26.37 |

### Example 4

The method of this example is essentially the same as that of sample C in Example 2, where the sample was extracted in two steps (two passes through the column), and the samples and reagents used were listed in Table 6 below.

TABLE 6

| | | | Quantity | | Pro- | standard of |
|---|---|---|---|---|---|---|
| | Speci- | | of | | teinase | pseudovirus |
| NO. | mens | Volume | Specimens | BB | K | of COVID-19 |
| A | saliva | 1.8 ml | 2 | 2.2 ml | 0.2 ml | 200 ul |
| B | saliva | 1.8 ml | 2 | 2.2 ml | 0.2 ml | 200 ul |
| C | saliva | 1.8 ml | 2 | 2.2 ml | 0.2 ml | 200 ul |
| D | saliva | 1.8 ml | 2 | 2.2 ml | 0.2 ml | 200 ul |

The results of each primer pair were shown in Table 7, where the meaning of the sample number in Table 7 is as follows, taking A2-1 as an example, where A refers to the serial number corresponding to the specimen in Table 6, 2 refers to the second specimen of that serial number, and −1 refers to the test solution collected by the first pass through the column, so A2-1 refers to the test result corresponding to the test solution collected by the first pass through the column for the second A sample.

TABLE 7

| | RT-qPCR CT | | | | | | | |
|---|---|---|---|---|---|---|---|---|
| Name | | | | | | | | E Gene |
| of | M11 | K11 | C5 | B9 | NCPN2 | P5 | RdRp-2 | of EU |
| sample | Cq | Cq | Cq | Cq | Cq | Cq | Cq | Cq |
| A2-1 | 33.13 | 26.28 | 33.76 | 26.84 | 32.84 | 27.29 | 32 | 30.17 |
| B1-1 | 28.84 | 24.02 | 28.48 | 25.21 | 34.96 | 26.15 | 32.4 | 27.36 |
| B2-1 | 31.09 | 25.07 | 33.68 | 28.5 | 31.98 | 26.45 | 34.12 | 32.02 |
| C1-1 | N/A | 23.62 | 32.54 | 27.78 | 39.21 | 25.21 | 33.7 | 31.18 |
| C2-1 | 31.61 | 24.42 | 32.72 | 29.05 | 32.9 | 26.26 | 31.43 | 30.15 |
| A2-2 | 28.99 | 21.45 | 32.75 | 28.49 | 30.56 | 23 | 30.06 | 32.06 |
| B2-2 | 28.93 | 21.46 | 32.42 | 29.47 | 32.24 | 23.11 | 32.33 | 35.11 |
| C2-2 | 29.74 | 21.37 | 45.54 | 28.31 | 31.19 | 23.45 | 32.37 | 34.41 |

where the upstream primer for RdRp-2 is gtgaratggtcatgtgtggcgg (SEQ TD NO. 125) and the downstream primer is caratgttaaasacactattagcata (SEQ ID NO.126).

The upstream primer for E Gene of EU is acaggtacgttaatagttaatagcgt (SEQ TD NO. 123) and the downstream primer is atattgcagcagtacgcacaca (SEQ ID NO. 124).

### Example 5

Comparison of sensitivity of Magnetic Bead Extraction Kit/PCR COVID-19 Reaction Kit (Daan/Sansure) and OBI product (MOES)

Daan/Sansure: 200 ul of pharyngeal swab extracted, about 50 ul of eluate was collected, 25 ul of reaction system at RT-qPCR, of which 5 ul of eluate (template) was used.

The OBI product (MOES) was extracted and detected as in Example 4.

The assay results are shown in Table 8.

TABLE 8

| Fluorescence | | | Fluorescence | | | |
|---|---|---|---|---|---|---|
| channel | Daan | Ct | channel | Sansure | Ct1 | Ct2 |
| Cy5 | internal | 25.83 | HEX | internal | 25 | 25 |
| | standard | | | standard | | |
| FAM | N gene | 34.79 | FAM | N gene | 36.94 | 36.78 |
| Yellow | ORF1ab | 35.75 | ROX | ORF1ab | 33.81 | 33.93 |
| (VIC) | | | | | | |

Comparison of dilution multiples

| | Daan | OBI | | Sansure |
|---|---|---|---|---|
| Volume of | 200 ul | 2 | ml | 200 ul |
| sample | | | | |
| Volume of | 60 ul | 60 | ul | 60 ul |
| eluate | | | | |

| | | | |
|---|---|---|---|
| qPCR | 5 ul/25 ul | 4 ul/20 ul | 20 ul/50 ul |

As seen from Table 7, the ct values of N gene for the OBI product of this application were 31-33, the internal standard ct values were 21-23, and the E gene ct values were 27-31; while the corresponding Ct values of Daan and Sansure were 3-4 higher than those of this application, thus, the sensitivity of OBI was more than 16 times higher than that of Daan/Sansure.

### Example 6

In this example, different samples were processed and tested according to the method of Example 2. Some sequences are shown in Table 9 and some sequences are shown in Table 13, and the test results of each sequence are shown in Table 10 to Table 16, respectively.

TABLE 9

| | | | | | | Amplified |
|---|---|---|---|---|---|---|
| | | | | | | fragment |
| Group | target | Primer name | | Primer sequence | Position | length |
| A | IP-10 | A1 | Upstream | gccttatctttctgactct (SED IQ | e1 | 107 |
| | | | primer | NO. 1) | | |
| | | | Downstream | taaagaccttggattaaca (SED IQ | e2 | |
| | | | primer | NO. 2) | | |
| | | A2 | Upstream | ctgccttatctttctgact (SED IQ | e1 | 109 |
| | | | primer | NO. 3) | | |
| | | | Downstream | taaagaccttggattaaca (SED IQ | e2 | |
| | | | primer | NO. 4) | | |
| | | A3 | Upstream | catcaagaatttactgaaag (SED IQ | e3 | 176 |
| | | | primer | NO. 5) | | |
| | | | Downstream | taactgcaaactaagaacaa (SED IQ | e4 | |
| | | | primer | NO. 6) | | |
| | | A4 | Upstream | caagaatttactgaaagca (SED IQ | e3 | 160 |
| | | | primer | NO. 7) | | |
| | | | Downstream | agaacaattatggcttgac (SED IQ | e4 | |
| | | | primer | NO. 8) | | |
| B | IL6 | B1 | Upstream | ccaggagcccagctatgaac (SED IQ | e1 | 62 |
| | | | primer | NO. 9) | | |
| | | | Downstream | cagggagaaggcaactggac (SED | e2 | |
| | | | primer | IQ NO. 10) | | |
| | | B2 | Upstream | acatcctcgacggcatctca (SED IQ | e2 | 75 |
| | | | primer | NO. 11) | | |
| | | | Downstream | cctctttgctgctttcacaca (SED IQ | e3 | |
| | | | primer | NO. 12) | | |
| | | B3 | Upstream | aacaacctgaaccttccaaaga (SED | e3 | 91 |
| | | | primer | IQ NO. 13) | | |
| | | | Downstream | cagtgatgattttcaccaggca (SED IQ | e4 | |
| | | | primer | NO. 14) | | |
| | | B4 | Upstream | gcccagctatgaactccttct (SED IQ | e1 | 61 |
| | | | primer | NO. 15) | | |
| | | | Downstream | gcggctacatctttggaatct (SED IQ | e2 | |
| | | | primer | NO. 16) | | |
| | | B5 | Upstream | gagcccagctatgaactcctt (SED IQ | e1 | 63 |
| | | | primer | NO. 17) | | |
| | | | Downstream | gcggctacatctttggaatct (SED IQ | e2 | |
| | | | primer | NO. 18) | | |
| | | B6 | Upstream | ccaggagcccagctatgaac (SED IQ | e1 | 69 |
| | | | primer | NO. 19) | | |
| | | | Downstream | gggcggctacatctttgga (SED IQ | e2 | |
| | | | primer | NO. 20) | | |
| | | B7 | Upstream | ccagagctgtgcagatgagt(SED IQ | e3 | 100 |
| | | | primer | NO. 21) | | |
| | | | Downstream | gcatttgtggttgggtcagg (SED IQ | e4 | |
| | | | primer | NO. 22) | | |
| | | B8 | Upstream | gagcccagctatgaactcctt (SED IQ | e1 | 63 |
| | | | primer | NO. 23) | | |
| | | | Downstream | tttctgccagtgcctctttg (SED IQ | e2 | |
| | | | primer | NO. 24) | | |
| C | IL2 | C1 | Upstream | acagtgcacctacttcaagtt (SED IQ | e1 | 120 |
| | | | primer | NO. 37) | | |
| | | | Downstream | tcctggtgagtttgggattct (SED IQ | e2 | |
| | | | primer | NO. 38) | | |
| | | C2 | Upstream | tgcacctacttcaagttctaca (SED IQ | e1 | 114 |
| | | | primer | NO. 39) | | |
| | | | Downstream | tcctggtgagtttgggattct (SED IQ | e2 | |
| | | | primer | NO. 40) | | |
| | | C3 | Upstream | gacccagggacttaatcagca (SED IQ | e3 | 95 |
| | | | primer | NO. 41) | | |
| | | | Downstream | tgctgtctcatcagcatattcac (SED | e4 | |
| | | | primer | IQ NO. 42) | | |
| D | IL17A | D1 | Upstream | ctcctgggaagacctcattg (SED IQ | e1 | 80 |
| | | | primer | NO. 47) | | |
| | | | Downstream | tgggattgtgattcctgcct (SED IQ | e2 | |
| | | | primer | NO. 48) | | |
| | | D2 | Upstream | tgactcctgggaagacctca (SED IQ | e1 | 71 |
| | | | primer | NO. 49) | | |
| | | | Downstream | cctgccttcactatggcctc (SED IQ | e2 | |
| | | | primer | NO. 50) | | |
| E | IL13 | E1 | Upstream | atggtatggagcatcaacctg (SED IQ | e3 | 95 |
| | | | primer | NO. 51) | | |
| | | | Downstream | tgggtcttctcgatggcact (SED IQ | e4 | |
| | | | primer | NO. 52) | | |
| | | E2 | Upstream | cagaggatgctgagcggatt (SED IQ | e4 | 86 |
| | | | primer | NO. 53) | | |
| | | | Downstream | acctcgattttggtgtctcgg (SED IQ | e5 | |
| | | | primer | NO. 54) | | |
| F | TNF | F1 | Upstream | ctgcactttggagtgatcgg (SED IQ | e1 | 71 |
| | | | primer | NO. 55) | | |
| | | | Downstream | ccagagggctgattagagag (SED IQ | e2 | |
| | | | primer | NO. 56) | | |
| | | F2 | Upstream | cttctgcctgctgcactttg (SED IQ | e1 | 78 |
| | | | primer | NO. 57) | | |
| | | | Downstream | agagggctgattagagagaggt (SED | e2 | |
| | | | primer | IQ NO. 58) | | |
| | | F3 | Upstream | cctctctctaatcagccctc (SED IQ | e2 | 73 |
| | | | primer | NO. 59) | | |
| | | | Downstream | gctacaggcttgtcactcgg (SED IQ | e3 | |
| | | | primer | NO. 60) | | |
| | | F4 | Upstream | gacctctctctaatcagcc (SED IQ | e2 | 70 |
| | | | primer | NO. 61) | | |
| | | | Downstream | ggcttgtcactcggggttc (SED IQ | e3 | |
| | | | primer | NO. 62) | | |
| | | F5 | Upstream | agcctgtagcccatgttgtag (SED IQ | e3 | 107 |
| | | | primer | NO. 63) | | |
| | | | Downstream | gttatctctcagctccacgcc (SED IQ | e4 | |
| | | | primer | NO. 64) | | |
| G | MCP-1 | G1 | Upstream | gctcatagcagccaccttcat (SED IQ | e1 | 104 |
| | | | primer | NO. 65) | | |
| | | | Downstream | gcactgagatcttcctattg (SED IQ | e2 | |
| | | | primer | NO. 66) | | |
| | | G2 | Upstream | cgcgagctatagaagaatcac (SED IQ | e2 | 99 |
| | | | primer | NO. 67) | | |
| | | | Downstream | ttctgcttggggtcagcac (SED IQ | e3 | |
| | | | primer | NO. 68) | | |
| H | HMOX1 | H1 | Upstream | gaactttcagaagggccaggt (SED IQ | e2 | 72 |
| | | | primer | NO. 69) | | |
| | | | Downstream | acatagatgtggtacagggagg (SED | e3 | |
| | | | primer | IQ NO. 70) | | |
| | | H2 | Upstream | gaactttcagaagggccaggt (SED IQ | e2 | 99 |
| | | | primer | NO. 71) | | |
| | | | Downstream | ttgcgctcaatctcctcctc (SED IQ | e3 | |
| | | | primer | NO. 72) | | |
| | | H3 | Upstream | ccaggcagagaatgctgagtt (SED IQ | e2 | 99 |
| | | | primer | NO. 73) | | |
| | | | Downstream | acatagatgtggtacagggagg (SED | e3 | |
| | | | primer | IQ NO. 74) | | |
| | | H4 | Upstream | gtcaggcagagggtgatagaa (SED | e3 | 99 |
| | | | primer | IQ NO. 75) | | |
| | | | Downstream | gtccttggtgtcatgggtcag (SED IQ | e4 | |
| | | | primer | NO. 76) | | |
| | | H5 | Upstream | agggtgatagaagaggccaag (SED | e3 | 67 |
| | | | primer | IQ NO. 77) | | |
| | | | Downstream | gctcctgcaactccctcaaag (SED IQ | e4 | |
| | | | primer | NO. 78) | | |
| | | H6 | Upstream | ggccagcaacaaagtgcaag (SED IQ | e4 | 106 |
| | | | primer | NO. 79) | | |
| | | | Downstream | gagtgtaaggacccatcggag (SED | e5 | |
| | | | primer | IQ NO. 80) | | |
| I | IFNγ | I1 | Upstream | caggtcattcagatgtagcgg (SED IQ | e2 | 158 |
| | | | primer | NO. 81) | | |
| | | | Downstream | actcttttggatgctctggtc (SED IQ | e3 | |
| | | | primer | NO. 82) | | |
| | | I2 | Upstream | cgagatgacttcgaaaagctga (SED | e3 | 71 |
| | | | primer | IQ NO. 83) | | |
| | | | Downstream | tcatgtattgctttgcgttgga (SED IQ | e4 | |
| | | | primer | NO. 84) | | |
| J | VWF | J1 | Upstream | gctgtgtggcaactttaacatc (SED IQ | e5 | 89 |
| | ID: | | primer | NO. 85) | | |
| | (Gene | | Downstream | agttggcaaagtcataagggtc (SED | e6 | |
| | ID: | | primer | IQ NO. 86) | | |
| | 7450) | J2 | Upstream | agtttcgccaaggctttcatt (SED IQ | e29 | 61 |
| | | | primer | NO. 87) | | |
| | | | Downstream | ctgacacctgagtgagacgag (SED | e30 | |
| | | | primer | IQ NO. 88) | | |
| | | J3 | Upstream | cctccagtttcccagcttctta (SED IQ | e29 | 114 |
| | | | primer | NO. 89) | | |
| | | | Downstream | ttccatactgcagcactgaca (SED IQ | e30 | |
| | | | primer | NO. 90) | | |
| K | SELP | K1 | Upstream | gacactggtctgcaccctt (SED IQ | e3 | 60 |
| | | | primer | NO. 91) | | |
| | | | Downstream | gactctccagcggctcaca (SED IQ | e4 | |
| | | | primer | NO. 92) | | |
| | | K2 | Upstream | ttgactctggacactggtctg (SED IQ | e7 | 90 |
| | | | primer | NO. 93) | | |
| | | | Downstream | aatccatgcttccgtggaca (SED IQ | e8 | |
| | | | primer | NO. 94) | | |
| | | K3 | Upstream | gctgcattgactctggacac (SED IQ | e7 | 75 |
| | | | primer | NO. 95) | | |
| | | | Downstream | gactctccagcggctcaca (SED IQ | e8 | |
| | | | primer | NO. 96) | | |
| L | THBD | L1 | Upstream | acgtggatgactgcatactgg (SED IQ | e1 | 95 |
| | | | primer | NO. 97) | | |
| | | | Downstream | gtcgtagttagggtagcagtgg (SED | e1 | |
| | | | primer | IQ NO. 98) | | |
| | | L2 | Upstream | acgtggatgactgcatactgg (SED IQ | e1 | 98 |
| | | | primer | NO. 99) | | |
| | | | Downstream | caggtcgtagttagggtagca (SED IQ | e1 | |
| | | | primer | NO. 100) | | |
| | | L3 | Upstream | ggacgtggatgactgcatact (SED IQ | e1 | 70 |
| | | | primer | NO. 101) | | |
| | | | Downstream | gaagccaccctgtgtgttga (SED IQ | e1 | |
| | | | primer | NO. 102) | | |
| M | SAA | M1 | Upstream | gggaactatgatgctgccaa (SED IQ | e3 | 103 |
| | | | primer | NO. 103) | | |
| | | | Downstream | ccgcaccatggccaaagaa (SED IQ | e4 | |
| | | | primer | NO. 104) | | |
| | | M2 | Upstream | ccaattacatcggctcagaca (SED IQ | e3 | 120 |
| | | | primer | NO. 105) | | |
| | | | Downstream | tctggatattctctctggcatc (SED IQ | e4 | |
| | | | primer | NO. 106) | | |
| N | CRP | N1 | Upstream | ggtcttgaccagcctctctc (SED IQ | e1 | 78 |
| | | | primer | NO. 107) | | |
| | | | Downstream | tccgactctttgggaaacaca (SED IQ | e2 | |
| | | | primer | NO. 108) | | |
| | | N2 | Upstream | ttcactgtgtgcctccacttc (SED IQ | e2 | 127 |
| | | | primer | NO. 109) | | |
| | | | Downstream | cgccttgcacttcatacttca (SED IQ | e3 | |
| | | | primer | NO. 110) | | |
| | | N3 | Upstream | agcctctcaaagccttcact (SED IQ | e2 | 150 |
| | | | primer | NO. 111) | | |
| | | | Downstream | tgaacacttcgccttgcact (SED IQ | e3 | |
| | | | primer | NO. 112) | | |
| O | N | NCPN2 | Upstream | ctttgctgctgcttgacaga (SED IQ | | 71 |
| | gene | | primer | NO. 115) | | |
| | | | Downstream | gccttgttgttgttggcctt (SED IQ | | |
| | | | primer | NO. 116) | | |
| | | NCPN3 | Upstream | aactcaagccttaccgcaga (SED IQ | | 61 |
| | | | primer | NO. 117) | | |
| | | | Downstream | tgcagcaggaagaagagtca (SED IQ | | |
| | | | primer | NO. 118) | | |
| | E | NCPE1 | Upstream | tacactagccatccttact (SED IQ | | 78 |
| | gene | | primer | NO. 119) | | |
| | | | Downstream | gaaggttttacaagactca (SED IQ | | |
| | | | primer | NO. 120) | | |
| | | NCPE2 | Upstream | gttacactagccatccttac (SED IQ | | 80 |
| | | | primer | NO. 121) | | |
| | | | Downstream | gaaggttttacaagactcac (SED IQ | | |
| | | | primer | NO. 122) | | |
| | ORFab | ORFab- | Upstream | aaataccagtggcttaccgca (SED IQ | | |
| | | 1 | primer | NO. 137) | | |
| | | | Downstream | gccaccagctcctttattacc (SED IQ | | |
| | | | primer | NO. 138) | | |
| | | ORFab- | Upstream | ggtagcagaactcgaaggca (SED IQ | | |
| | | 2 | primer | NO. 139) | | |
| | | | Downstream | atgagggacaaggacaccaag (SED | | |
| | | | primer | IQ NO. 140) | | |
| | | ORFab- | Upstream | tccctgacttaaatggtgatgtg (SED | | |
| | | 3 | primer | IQ NO. 141) | | |
| | | | Downstream | tcttaaaagagggtgtgtagtgt (SED | | |
| | | | primer | IQ NO. 142) | | |

TABLE 10

| | | Sample number | | |
|---|---|---|---|---|
| | Primer | T2-1 | T4-1 | Blank control |
| target | name | Cq | Cq | Cq |
| IL6 | B1 | N/A | 33.18 | N/A |
| | B2 | N/A | N/A | N/A |
| | B3 | N/A | N/A | N/A |
| | B5 | 35.24 | 33.86 | N/A |
| | B7 | N/A | N/A | N/A |
| | B8 | N/A | N/A | N/A |
| HMOX1 | H1 | 35.66 | N/A | N/A |
| | H5 | 35.16 | 35.7 | N/A |

TABLE 11

| | | Sample number | | | |
|---|---|---|---|---|---|
| | Primer | RT1 | RT2 | RT3 | Blank control |
| target | name | Cq | Cq | Cq | Cq |
| IP-10 | A1 | N/A | N/A | N/A | N/A |
| | A4 | N/A | N/A | N/A | N/A |
| IL6 | B4 | 35.11 | 30.43 | 32.15 | N/A |
| | B6 | 32.97 | 31.74 | 31.74 | N/A |
| IL17A | D2 | N/A | 43.78 | N/A | N/A |
| HMOX1 | H4 | N/A | 33.58 | N/A | N/A |
| | H6 | N/A | 34.17 | N/A | N/A |
| IFNG | I2 | N/A | N/A | 34.50 | N/A |

TABLE 12

| | | Sample number | | |
|---|---|---|---|---|
| | Primer | T1-2 | T4-1 | Blank control |
| target | name | Cq | Cq | Cq |
| IL17A | D1 | 35.44 | N/A | N/A |
| | D2 | N/A | N/A | N/A |
| IL13 | E1 | 30.29 | 33.12 | N/A |
| | E2 | N/A | N/A | N/A |
| TNF | F1 | 33.06 | 38.45 | N/A |
| | F3 | 34.27 | 34.8 | N/A |
| | F4 | 36.22 | 39.37 | N/A |
| | F5 | 33.16 | 34.20 | N/A |

The sample numbers in Tables 10 to 11 above represent the samples as follows: T2-1 is the test result of the first pass-column collected test solution for the serum sample, T4-1 is the test result of the first pass-column collected test solution for the urine sample, RT1 is the test result of the serum sample, RT2 is the test result of the serum sample, RT3 is the test result of the nasopharyngeal swab sample, and T1-2 is the blood T1-2 are the results of the test solution collected from the second pass of the sample.

TABLE 13

| Primer | | 1 | | 2 | | |
|---|---|---|---|---|---|---|
| name | Primer sequence | Ct | Tm | Ct | Tm | target |
| P1 | Catgtgtggcggttcactat (SEQ ID | 39.41 | 76.0 | 35.68 | 79.5 | RNA |
| | NO. 129) | | | | | polymerase 2 |
| | tgcattaacattggccgtga (SEQ ID | | | | | |
| | NO. 130) | | | | | |
| P2 | ctacatgcaccagcaactgt (SEQ ID | N/A | | 45.26 | | RNA |
| | NO. 131) | | | | | polymerase 2 |
| | cacctgtgcctgttaaacca (SEQ ID | | | | | |
| | NO. 132) | | | | | |
| P3 | caatgctgcaatcgtgctac (SEQ ID | 39.33 | 82.5 | 35.06 | 82.5 | RNA |
| | NO. 133) | | | | | polymerase 2 |
| | gttgcgactacgtgatgagg (SEQ ID | | | | | |
| | NO. 134) | | | | | |
| P4 | gagatctctcaacgtgctcag (SEQ ID | 41.74 | | 41.01 | | RNA |
| | NO. 135) | | | | | polymerase 2 |
| | cttggcataaaacaggttcagaa (SEQ | | | | | |
| | ID NO. 136) | | | | | |
| L1 | SEQ ID NO. 97 | 21.87 | 82.5 | 22.06 | 82.5 | THBD |
| | SEQ ID NO. 98 | | | | | |
| L2 | SEQ ID NO. 99 | 21.96 | 82.5 | N/A | | THBD |
| | SEQ ID NO. 100 | | | | | |
| L3 | SEQ ID NO. 101 | 22.03 | 81.5 | 22.05 | 81.5 | THBD |
| | SEQ ID NO. 102 | | | | | |
| N3 | SEQ ID NO. 111 | 22.09 | 83.0 | 21.86 | 83.0 | CRP |
| | SEQ ID NO. 112 | | | | | |
| A2 | SEQ ID NO. 3 | 38.0 | 81.5 | N/A | | IP-10 |
| | SEQ ID NO. 4 | | | | | |

TABLE 14

| Primer | Primer | 1 | | 2 | | |
|---|---|---|---|---|---|---|
| name | sequence | Ct | Tm | Ct | Tm | target |
| A3 | SED IQ NO. 5 | N/A | | N/A | | IP-10 |
| | SED IQ NO. 6 | | | | | |
| C1 | SED IQ NO. 37 | 21.23 | 74.5 | 21.36 | 74.5 | IL2 |
| | SED IQ NO. 38 | | | | | |
| C2 | SED IQ NO. 39 | 20.71 | 74.5 | 21.20 | 74.5 | IL2 |
| | SED IQ NO. 40 | | | | | |
| J3 | SED IQ NO. 89 | 21.13 | 81.0 | 21.26 | 81.0 | VWF |
| | SED IQ NO. 90 | | | | | |
| K11 | SED IQ NO. 143 | 22.03 | 83.0 | 35.34 | 83.0 | Housekeeping |
| | SED IQ NO. 144 | | | | | Genes |
| K2 | SED IQ NO. 93 | 37.04 | 72.0 | 42.54 | 72.0 | SELP |
| | SED IQ NO. 94 | | | | | |
| K3 | SED IQ NO. 95 | 34.34 | 84.5 | 36.27 | 84.5 | SELP |
| | SED IQ NO. 96 | | | | | |
| J1 | SED IQ NO. 85 | N/A | | N/A | | VWF |
| | SED IQ NO. 86 | | | | | |

TABLE 15

| Primer | Primer | 1 | | 2 | | |
|---|---|---|---|---|---|---|
| name | sequence | Ct | Tm | Ct | Tm | target |
| C3 | SED IQ NO. 41 | N/A | | N/A | | IL2 |
| | SED IQ NO. 42 | | | | | |
| F2 | SED IQ NO. 57 | 22.87 | 82.5 | 22.79 | 82.5 | TNF |
| | SED IQ NO. 58 | | | | | |
| G1 | SED IQ NO. 65 | 25.13 | 83.0 | 24.33 | 83.0 | MCP-1 |
| | SED IQ NO. 66 | | | | | |
| G2 | SED IQ NO. 67 | 21.74 | 83.0 | 22.10 | 83.0 | MCP-1 |
| | SED IQ NO. 68 | | | | | |
| J2 | SED IQ NO. 87 | 22.00 | 80.5 | 20.99 | 80.5 | VWF |
| | SED IQ NO. 88 | | | | | |
| M11 | SEQ ID NO. 127 | 28.90 | 79.0 | 30.04 | 79.0 | BETA |
| | SEQ ID NO. 128 | | | | | MICRO- |
| | | | | | | GLOBULIN-2 |

TABLE 16

| | Test 1: nasopharyngeal | Test 2: nasopharyngeal |
|---|---|---|
| Primer | swabs + pseudovirus | swabs + pseudovirus |
| name | of COVID-19 (Ct) | of COVID-19 (Ct) |
| ORFab-2 | 26.66 | 24.16 |
| ORFab-3 | 27.83 | 23.03 |

The comparison between the conventional art and the technique of the embodiments in terms of virus collection and viral RNA extraction is shown in Table 17.

TABLE 17

| NO | conventional art | | | MOES | | | | | |
|---|---|---|---|---|---|---|---|---|---|
| 1 | Nasopharyngeal | | | Saliva/Nasopharyngeal | | | Urine collection | | |
| | swab collection | | | swab collection | | | | | |
| 2 | medical personnel | | | Self-collection | | | Self-collection | | |
| 3 | Preservation solution | 200-300 | ul | Saliva | 1-2 | ml | Urine | 2-20 | ml |
| 4 | Laboratory operation | | | Can be operated | | | Can be operated | | |
| | | | | on site (POCT) | | | on site (POCT) | | |
| 5 | Lysis, extraction | 500 | ul | Lysis, extraction | 4-16 | ml | Lysis, extraction | 4-16 | ml |
| 6 | Magnetic beads or | | | adsorption column | | | adsorption column | | |
| | adsorption column | | | | | | | | |
| 7 | Eluent volume | 60 | ul | Eluent volume | 60 | ul | Eluent volume | 60 | ul |

Advantage: 1. Using MOES with the feature of extracting a larger volume of body fluid specimen, a larger volume (10-40 ml) of water or saline is used to gargle the throat area to make the water or saline mix with saliva to extract RNA. 2. Cross-intron primer design, only the cDNA transcribed by mRNA after splicing is amplified, and the introns in the sequence have been cut out, not amplifying genomic DNA (gDNA), therefore, avoiding gDNA interference. Overall, it greatly improves sensitivity and specificity of the assay.

The reagents used in the process of sample collection, concentration, nucleic acid extraction, etc. may be sold separately or assembled into kits for sale.

Although the present invention has been disclosed in the form of embodiments and variations thereon, it will be understood that numerous additional modifications and variations could be made thereto without departing from the scope of the invention.

For the sake of clarity, it is to be understood that the use of ‘a’ or ‘an’ throughout this application does not exclude a plurality, and ‘comprising’ does not exclude other steps or elements.

1. A method of preparing a test solution for pathogen detection using a sample to be tested comprising:
lysing the sample to be tested with a lysis buffer to release the nucleic acids contained in the sample to obtain a lysis buffer containing nucleic acids, the nucleic acids include host nucleic acids, and/or when the sample to be tested contains pathogens, the nucleic acids also include pathogen nucleic acids; wherein the host nucleic acids include nucleic acids of one or more of cytokines, chemokines, and biomarkers from human or other mammals,
extracting the lysis buffer containing nucleic acids through a nucleic acid extraction device to obtain an extract containing host nucleic acids, and/or when the sample to be tested contains pathogens, the extract also contains pathogen nucleic acids;
preparing the test solution for pathogen detection and for host nucleic acids detection from the extract;
wherein the method also includes a step A:
A. controlling the environment where the extract is located to stabilize the nucleic acids in the extract so that the test solution contains the nucleic acids; wherein an oxygen content in the environment where the extract is located is controlled to be less than 1%; and
wherein the method also includes a step selected from at least one of the following:
B. making the lysis buffer and/or washing buffer used in the extraction process contain tris(2-carboxyethyl)phosphine hydrochloride;
C. controlling the volume of the sample to be tested to 1-40 mL; and
D. using a sealed nucleic acid extraction device for the extraction.

2. The method of claim 1, wherein in the step A, an oxygen content in an environment where the extract is located is controlled to be about 0.01%˜1%; and/or, the oxygen content can be controlled by adding oxygen scavengers to the environment; and/or, the method comprises adding oxygen scavengers to the environment where the extract is located before the start of extraction, during the extraction process, or after the extraction.

3. The method of claim 1, wherein; the extraction step includes passing the lysis buffer containing nucleic acids through a filter column for adsorption, and then passing a washing buffer for washing and then eluting the filter column with an eluent to obtain a test solution, where at least the adsorption and washing are performed in a sealed environment.

4. The method of claim 1, wherein the extraction device comprises:
a receptacle defining an internal volume;
a removable cap for the receptacle, the cap having an internal side facing the internal volume of the receptacle and an external side facing away from the internal volume, the cap comprising a breather port communicating between the internal side and the external side and a sample connection port communicating between the internal side and the external side, the sample connection port comprising a first interlocking component for releasably locking the sample connection port to a cooperating second interlocking component, the internal side of the cap comprising a connection interface in fluid communication with the sample connection port;
a filter column configured to be removably attached to the connection interface of the receptacle cap, the filter column having an open first end, an open second end, and an internal passage therebetween containing a substrate for collecting the nucleic acid; and
a shipping container having an open end and defining a volume configured to contain the filter column, the shipping container configured to releasably engage the filter column for detaching it from the connection interface of the receptacle cap, the shipping container further comprising a removable lid for temporarily sealing the filter column within the shipping container.

5. The method of claim 1, wherein passing the lysis buffer containing nucleic acids through a treated filter column for adsorption of nucleic acids; or mixing the lysis buffer containing nucleic acids with a binding buffer and then passing through the filter column for adsorption.

6. The method of claim 1, wherein the method further comprises the step of collecting the sample to be tested using a collection device; the collection device comprising a sample collector and a sample storage container detachably interconnected with the sample collector;
the sample collector comprises a connecting part that is detachably interconnected with the sample storage container, a collecting part fixedly connected to the connecting part at one end or integrally formed with the connecting part at one end and having an opening, the cross-sectional area of the opening of the collecting part is larger than the cross-sectional area of the connecting position where the collecting part is connected;
the cross-section of the opening of the collecting part is circular or oval;
the sample collector and the sample storage container are threadedly connected;
the connecting part comprises a first part having an internal passage communicating with the collecting part, and a second part fixedly arranged outside the first part; the first part is fixedly connected with the lower part of the collecting part or the first part is integrally formed with the lower part of the collecting part; an accommodating space is formed between the inner wall of the second part and the outer wall of the first part; an internal thread is formed on the inner wall of the second part, an external thread is formed on the outer wall of the sample storage container; when the sample collector is connected to the sample storage container, the sample storage container is connected with the second part and a gap is formed between the inner wall of the sample storage container and the outer wall of the first part;
the sample storage container is added or pre-stored with sample processing substances used to inactivate, preserve, digest or release the nucleic acids in the sample;
the sample processing substances is liquid or solid; and
the sample processing substances is in a dry powder state.

7. The method of claim 1, wherein the sample to be tested includes but is not limited to blood, body fluids, secretions, and/or excrement; and/or wherein the sample to be tested is one or more of saliva, urine, nasopharyngeal swab, oropharyngeal swab, bronchial/lung lavage, cerebrospinal fluid, lymphatic fluid, ascites, amniotic fluid, peritoneal dialysis fluid, and/or,
wherein the volume of the sample to be tested is 2-20 ml; and/or,
the cytokines, chemokines or biomarkers are cytokines, chemokines or biomarkers produced by the host after the pathogens enter the host; and/or wherein the cytokines include but are not limited to one or more of IL1B, IL1RA, IL2, IL3, IL4, IL5, IL6, IL7, IL8, IL9, IL10, IL12p70, IL13, IL15, IL17A, IL23, IL25, IL27 and IL33, the chemokines include but are not limited to one or more of chemokines CCL1, CCL2, CCL3, CCL11, CXCL1, CXCL2, CXCL8, CXCL9, CXCL10, and CXCL11 and eosinophil-activated chemokine, the biomarkers include but are not limited to one or more of basic FGF2, CSF, GCSF, GMCSF, IFN, IFNγ, IP-10, MCP1, MIP1A, MIP1B, PDGFB, RANTES, TNF, TGFβ, TSLP, VEGFA, HO1, CRP, PCT, SAA, vWF, SELP and THBD; or the cytokines include but are not limited to one or two or three or four or five of IL2, IL6, IL10, IL17A and IL13, or the biomarkers include but are not limited to one or two or three or four or more than four of HO1, CRP, IP-10, SAA, TNF, MCP1, IFNγ, vWF, SELP and THBD; further wherein the biomarkers at least include HO1; further wherein the cytokines include one or more of IL2, IL6 and IL10, the biomarkers include one or two of HO1, CRP and IP-10, and SAA; and/or,
nucleic acids from a host include free nucleic acid, and RNA, mRNA and DNA; and/or,
the washing buffer further contains absolute ethanol; and/or,
the concentration of the tris(2-carboxyethyl)phosphine hydrochloride in the lysis buffer or the washing buffer is 1 to 20 mM.

8. The method of claim 1, wherein the pathogens include but are not limited to viruses, bacteria, fungi or parasites and/or wherein the pathogen is COVID-19.

9. A preparation system of preparing a test solution for pathogen detection using a sample to be tested comprising an extraction device and collection device:
the extraction device comprising:
a receptacle defining internal volume;
a removable cap for the receptacle, the call having an internal side facing the internal volume of the receptacle and an external side facing away from the internal volume, the cap comprising a breather port communicating between the internal side and the external side and a sample connection port communicating between the internal side and the external side, the sample connection port comprising a first interlocking component for releasably locking the sample connection port to a cooperating second interlocking component, the internal side of the cap comprising a connection interface in fluid communication with the sample connection port;
a filter column configured to be removably attached to the connection interface of the receptacle cap, the filter column having an open first end, an open second end, and an internal passage therebetween containing a substrate for collecting the nucleic acid;
a shipping container having an open end and defining a volume configured to contain the filter column, the shipping container configured to releasably engage the filter column for detaching it from the connection interface of the receptacle cap, the shipping container further comprising a removable lid for temporarily sealing the filter column within the shipping container;
the collection device comprising:
a sample collector and a sample storage container detachably interconnected with the sample collector;
a connecting part that is detachably interconnected with the sample storage container, a collecting part fixedly connected to the connecting part at one end or integrally formed with the connecting part at one end and having an opening, the cross-sectional area of the opening of the collecting part is larger than the cross-sectional area of the connecting position where connected to the collectin part;
wherein the cross section of the opening of the collecting par is circular or oval;
wherein the sample collector and the ample storage container are threadedly connected;
wherein the connecting part comprises a first part having an internal passage communicating with the collecting part, and a second part fixedly arranged outside the first part; the first part is fixedly connected with the lower part of the collecting part or the first part is integrally formed with the lower part of the collecting part; an accommodating space is formed between the inner wall of the second part and the outer wall of the first part; an internal thread is formed on the inner wall of the second part, an external thread is formed on the outer wall of the sample storage container; when the sample collector is connected to the sample storage container, the sample storage container is connected with the second part and a gap is formed between the inner wall of the sample storage container and the outer wall of the first part; and
wherein the sample storage container is added or pre-stored with sample processing substances used to inactivate, preserve, digest or release the nucleic acids in the sample.

10. A kit of preparing a test solution for pathogen detection using a sample to be tested comprising one or more of a washing buffer, a sample processing solution, a deoxidizer, a lysis buffer, a binding buffer, an elution buffer and a protease used in the method of claim 1.

11. The method of claim 1, wherein the pathogens include one or more of viruses, bacteria, fungi, parasites, and the host includes human or other mammals.

12. Primers for detecting the nucleic acids of the COVID 19 and the host nucleic acids, wherein the primers include primer pairs targeting the nucleic acids of COVID-19, and primer pairs targeting one or more of the host nucleic acids.

13. The primers of claim 12, wherein nucleic acids from a host include, but are not limited to, nucleic acids of one or more of cytokines, chemokines, and biomarkers; and/or wherein the cytokines, chemokines or biomarkers are cytokines, chemokines or biomarkers produced by the host after the pathogens enter the host; and/or wherein the cytokines, chemokines or biomarkers include but are not limited to one or more of IL1B, IL1RA, IL2, IL3, IL4, IL5, IL6, IL7, IL8, IL9, IL10, IL12p70, IL13, IL15, IL17A, IL23, IL25, IL27 and IL33, the chemokines include but are not limited to one or more of chemokines CCL1, CCL2, CCL3, CCL11, CXCL1, CXCL2, CXCL8, CXCL9, CXCL10 and CXCL11 and eosinophil-activated chemokine, the biomarkers include but are not limited to one or more of basic FGF2, CSF, GCSF, GMCSF, IFN, IFNγ, IP-10, MCP1, MIP1A, MIP1B, PDGFB, RANTES, TNF, TGFβ, TSLP, VEGFA, HO1, CRP, PCT, SAA, vWF, SELP and THBD; or the cytokines include but are not limited to one or two or three or four or five of IL2, IL6, IL10, IL17A and IL13, or the biomarkers include but are not limited to one or two or three or four or more than four of HO1, CRP, IP-10, SAA, TNF, MCP1, IFNγ, vWF, SELP and THBD; further the biomarkers at least include HO1; further the cytokines include one or more of IL2, IL6 and IL10, the biomarkers include one or two of HO1, CRP and IP-10, and SAA; and/or,
the primer pairs targeting the nucleic acids of COVID-19 are one or more of the primer pairs targeting the E gene of COVID-19, and/or one or more of the primer pairs targeting the N gene of COVID-19, and/or one or more of the primer pairs targeting the RdRp gene of COVID-19; and/or one or more of the primer pairs targeting the ORFab gene of COVID-19;
the sequences of the primer pairs targeting the E gene are one, more than one or all of the primer pairs of SEQ ID NO. 119 to 124;
the sequences of the primer pairs targeting the N gene are one, more than one or all of the primer pairs of SEQ ID NO. 27-28, 31-36, 115 to 118;
the sequences of the primer pairs targeting the RdRp gene are one or two of the primer pairs of SEQ ID NO. 113-114, 125 to 126;
the sequences of the primer pairs targeting the ORFab gene are one, more than one or all of the primer pairs of SEQ ID NO. 137 to 142;
the primer pairs targeting the nucleic acids from a host are one or more of the primer pairs targeting IP-10, the primer pairs targeting IL6, the primer pairs targeting IL2, the primer pairs targeting IL17A, the primer pairs targeting IL13, the primer pairs targeting TNF, the primer pairs targeting MCP1, the primer pairs targeting HO1, the primer pairs targeting IFNγ, the primer pairs targeting vWF, the primer pairs targeting SELP, the primer pairs targeting THBD, the primer pairs targeting SAA and the primer pairs targeting CRP;
the sequences of the primer pairs targeting the IP-10 are one, more than one or all of the primer pairs of SEQ ID NO. 1 to 8;
the sequences of the primer pairs targeting the IL6 are one, more than one or all of the primer pairs of SEQ ID NO. 9 to 24;
the sequences of the primer pairs targeting the IL2 are one, more than one or all of the primer pairs of SEQ ID NO. 37 to 46;
the sequences of the primer pairs targeting the IL17A are one, or all of the primer pairs of SEQ ID NO. 47 to 50;
the sequences of the primer pairs targeting the IL13 are one, more than one or all of the primer pairs of SEQ ID NO. 51 to 54;
the sequences of the primer pairs targeting the TNF are one, more than one or all of the primer pairs of SEQ ID NO. 55 to 64;
the sequences of the primer pairs targeting the MCP1 are one, or all of the primer pairs of SEQ ID NO. 65 to 68;
the sequences of the primer pairs targeting the HO1 are one, more than one or all of the primer pairs of SEQ ID NO. 69 to 80;
the sequences of the primer pairs targeting the IFNγ are one, more than one or all of the primer pairs of SEQ ID NO. 81 to 84;
the sequences of the primer pairs targeting the vWF are one, more than one or all of the primer pairs of SEQ ID NO. 85 to 90;
the sequences of the primer pairs targeting the SELP are one, more than one or all of the primer pairs of SEQ ID NO. 91 to 96;
the sequences of the primer pairs targeting the THBD are one, more than one or all of the primer pairs of SEQ ID NO. 97 to 102;
the sequences of the primer pairs targeting the SAA are one, more than one or all of the primer pairs of SEQ ID NO. 103 to 106; and/or
the sequences of the primer pairs targeting the CRP are one, more than one or all of the primer pairs of SEQ ID NO. 107 to 112.

14. The primers of claim 12, wherein the primers include primer pairs targeting the nucleic acids of housekeeping genes of host, the sequences of the primer pairs are one, more than one or all of the primer pairs of SEQ ID NO. 29-30, 127 to 136, 143-144.

15. A nucleic acid detection method using a test solution prepared by the method of claim 1, comprising using primers to perform quantitative PCR detection on the test solution, and the test solution contains host nucleic acids; the primers include primer pairs targeting the nucleic acids of pathogens, and primer pairs targeting one or more of the host nucleic acids;
the pathogens include but are not limited to COVID-19;
nucleic acids from a host include, but are not limited to, nucleic acids of one or more of cytokines, chemokines, and biomarkers; and/or wherein the cytokines, chemokines or biomarkers are cytokines, chemokines or biomarkers produced by the host after the pathogens enter the host; and/or wherein the cytokines, chemokines or biomarkers include but are not limited to one or more of IL1B, IL1RA, IL2, IL3, IL4, IL5, IL6, IL7, IL8, IL9, IL10, IL12p70, IL13, IL15, IL17A, IL23, IL25, IL27 and IL33, the chemokines include but are not limited to one or more of chemokines CCL1, CCL2, CCL3, CCL11, CXCL1, CXCL2, CXCL8, CXCL9, CXCL10 and CXCL11 and eosinophil-activated chemokine, the biomarkers include but are not limited to one or more of basic FGF2, CSF, GCSF, GMCSF, IFN, IFNγ, IP-10, MCP1, MIP1A, MIP1B, PDGFB, RANTES, TNF, TGFβ, TSLP, VEGFA, HO1, CRP, PCT, SAA, vWF, SELP and THBD; or the cytokines include but are not limited to one or two or three or four or five of IL2, IL6, IL10, IL17A and IL13, or the biomarkers include but are not limited to one or two or three or four or more than four of HO1, CRP, IP-10, SAA, TNF, MCP1, IFNγ, vWF, SELP and THBD; further the biomarkers at least include HO1; further the cytokines include one or more of IL2, IL6 and IL10, the biomarkers include one or two of HO1, CRP and IP-10, and SAA; and/or,
the host nucleic acid includes free nucleic acid, and RNA, mRNA and DNA; and
nucleic acids from a host are mRNA.


SEQUENCE LISTING (144 sequences)

SEQ ID NO: 1 - DNA, 19 bp, Artificial Sequence
  the sequence is synthesized
gccttatctttctgactct

SEQ ID NO: 2 - DNA, 19 bp, Artificial Sequence
  the sequence is synthesized
taaagaccttggattaaca

SEQ ID NO: 3 - DNA, 19 bp, Artificial Sequence
  the sequence is synthesized
ctgccttatctttctgact

SEQ ID NO: 4 - DNA, 19 bp, Artificial Sequence
  the sequence is synthesized
taaagaccttggattaaca

SEQ ID NO: 5 - DNA, 20 bp, Artificial Sequence
  the sequence is synthesized
catcaagaatttactgaaag

SEQ ID NO: 6 - DNA, 20 bp, Artificial Sequence
  the sequence is synthesized
taactgcaaactaagaacaa

SEQ ID NO: 7 - DNA, 19 bp, Artificial Sequence
  the sequence is synthesized
caagaatttactgaaagca

SEQ ID NO: 8 - DNA, 19 bp, Artificial Sequence
  the sequence is synthesized
agaacaattatggcttgac

SEQ ID NO: 9 - DNA, 20 bp, Artificial Sequence
  the sequence is synthesized
ccaggagcccagctatgaac

SEQ ID NO: 10 - DNA, 20 bp, Artificial Sequence
  the sequence is synthesized
cagggagaaggcaactggac

SEQ ID NO: 11 - DNA, 20 bp, Artificial Sequence
  the sequence is synthesized
acatcctcgacggcatctca

SEQ ID NO: 12 - DNA, 21 bp, Artificial Sequence
  the sequence is synthesized
cctctttgctgctttcacaca

SEQ ID NO: 13 - DNA, 22 bp, Artificial Sequence
  the sequence is synthesized
aacaacctgaaccttccaaaga

SEQ ID NO: 14 - DNA, 22 bp, Artificial Sequence
  the sequence is synthesized
cagtgatgattttcaccaggca

SEQ ID NO: 15 - DNA, 21 bp, Artificial Sequence
  the sequence is synthesized
gcccagctatgaactccttct

SEQ ID NO: 16 - DNA, 21 bp, Artificial Sequence
  the sequence is synthesized
gcggctacatctttggaatct

SEQ ID NO: 17 - DNA, 21 bp, Artificial Sequence
  the sequence is synthesized
gagcccagctatgaactcctt

SEQ ID NO: 18 - DNA, 21 bp, Artificial Sequence
  the sequence is synthesized
gcggctacatctttggaatct

SEQ ID NO: 19 - DNA, 20 bp, Artificial Sequence
  the sequence is synthesized
ccaggagcccagctatgaac

SEQ ID NO: 20 - DNA, 19 bp, Artificial Sequence
  the sequence is synthesized
gggcggctacatctttgga

SEQ ID NO: 21 - DNA, 20 bp, Artificial Sequence
  the sequence is synthesized
ccagagctgtgcagatgagt

SEQ ID NO: 22 - DNA, 20 bp, Artificial Sequence
  the sequence is synthesized
gcatttgtggttgggtcagg

SEQ ID NO: 23 - DNA, 21 bp, Artificial Sequence
  the sequence is synthesized
gagcccagctatgaactcctt

SEQ ID NO: 24 - DNA, 20 bp, Artificial Sequence
  the sequence is synthesized
tttctgccagtgcctctttg

SEQ ID NO: 25 - DNA, 21 bp, Artificial Sequence
  the sequence is synthesized
tacatcctcgacggcatctca

SEQ ID NO: 26 - DNA, 21 bp, Artificial Sequence
  the sequence is synthesized
gcctctttgctgctttcacac

SEQ ID NO: 27 - DNA, 21 bp, Artificial Sequence
  the sequence is synthesized
ttcggaagagacaggtacgtt

SEQ ID NO: 28 - DNA, 20 bp, Artificial Sequence
  the sequence is synthesized
cacacaatcgatgcgcagta

SEQ ID NO: 29 - DNA, 21 bp, Artificial Sequence
  the sequence is synthesized
tgagggcactggaaattgtat

SEQ ID NO: 30 - DNA, 23 bp, Artificial Sequence
  the sequence is synthesized
ctattaatgtggcgattgaccga

SEQ ID NO: 31 - DNA, 22 bp, Artificial Sequence
  the sequence is synthesized
ggtcatgtgtggcggttcacta

SEQ ID NO: 32 - DNA, 22 bp, Artificial Sequence
  the sequence is synthesized
gcataagcagttgtggcatctc

SEQ ID NO: 33 - DNA, 22 bp, Artificial Sequence
  the sequence is synthesized
caggcacaggtgttcttactga

SEQ ID NO: 34 - DNA, 21 bp, Artificial Sequence
  the sequence is synthesized
tgccaaattgttggaaaggca

SEQ ID NO: 35 - DNA, 20 bp, Artificial Sequence
  the sequence is synthesized
caatgctgcaatcgtgctac

SEQ ID NO: 36 - DNA, 21 bp, Artificial Sequence
  the sequence is synthesized
tgccgcctctgctcccttctg

SEQ ID NO: 37 - DNA, 21 bp, Artificial Sequence
  the sequence is synthesized
acagtgcacctacttcaagtt

SEQ ID NO: 38 - DNA, 21 bp, Artificial Sequence
  the sequence is synthesized
tcctggtgagtttgggattct

SEQ ID NO: 39 - DNA, 22 bp, Artificial Sequence
  the sequence is synthesized
tgcacctacttcaagttctaca

SEQ ID NO: 40 - DNA, 21 bp, Artificial Sequence
  the sequence is synthesized
tcctggtgagtttgggattct

SEQ ID NO: 41 - DNA, 21 bp, Artificial Sequence
  the sequence is synthesized
gacccagggacttaatcagca

SEQ ID NO: 42 - DNA, 23 bp, Artificial Sequence
  the sequence is synthesized
tgctgtctcatcagcatattcac

SEQ ID NO: 43 - DNA, 22 bp, Artificial Sequence
  the sequence is synthesized
ccaaactcaccaggatgctcac

SEQ ID NO: 44 - DNA, 22 bp, Artificial Sequence
  the sequence is synthesized
ccagaggtttgagttcttcttc

SEQ ID NO: 45 - DNA, 21 bp, Artificial Sequence
  the sequence is synthesized
gacccagggacttaatcagca

SEQ ID NO: 46 - DNA, 23 bp, Artificial Sequence
  the sequence is synthesized
tgtctcatcagcatattcacaca

SEQ ID NO: 47 - DNA, 20 bp, Artificial Sequence
  the sequence is synthesized
ctcctgggaagacctcattg

SEQ ID NO: 48 - DNA, 20 bp, Artificial Sequence
  the sequence is synthesized
tgggattgtgattcctgcct

SEQ ID NO: 49 - DNA, 20 bp, Artificial Sequence
  the sequence is synthesized
tgactcctgggaagacctca

SEQ ID NO: 50 - DNA, 20 bp, Artificial Sequence
  the sequence is synthesized
cctgccttcactatggcctc

SEQ ID NO: 51 - DNA, 21 bp, Artificial Sequence
  the sequence is synthesized
atggtatggagcatcaacctg

SEQ ID NO: 52 - DNA, 20 bp, Artificial Sequence
  the sequence is synthesized
tgggtcttctcgatggcact

SEQ ID NO: 53 - DNA, 20 bp, Artificial Sequence
  the sequence is synthesized
cagaggatgctgagcggatt

SEQ ID NO: 54 - DNA, 21 bp, Artificial Sequence
  the sequence is synthesized
acctcgattttggtgtctcgg

SEQ ID NO: 55 - DNA, 20 bp, Artificial Sequence
  the sequence is synthesized
ctgcactttggagtgatcgg

SEQ ID NO: 56 - DNA, 20 bp, Artificial Sequence
  the sequence is synthesized
ccagagggctgattagagag

SEQ ID NO: 57 - DNA, 20 bp, Artificial Sequence
  the sequence is synthesized
cttctgcctgctgcactttg

SEQ ID NO: 58 - DNA, 22 bp, Artificial Sequence
  the sequence is synthesized
agagggctgattagagagaggt

SEQ ID NO: 59 - DNA, 20 bp, Artificial Sequence
  the sequence is synthesized
cctctctctaatcagccctc

SEQ ID NO: 60 - DNA, 20 bp, Artificial Sequence
  the sequence is synthesized
gctacaggcttgtcactcgg

SEQ ID NO: 61 - DNA, 19 bp, Artificial Sequence
  the sequence is synthesized
gacctctctctaatcagcc

SEQ ID NO: 62 - DNA, 19 bp, Artificial Sequence
  the sequence is synthesized
ggcttgtcactcggggttc

SEQ ID NO: 63 - DNA, 21 bp, Artificial Sequence
  the sequence is synthesized
agcctgtagcccatgttgtag

SEQ ID NO: 64 - DNA, 21 bp, Artificial Sequence
  the sequence is synthesized
gttatctctcagctccacgcc

SEQ ID NO: 65 - DNA, 21 bp, Artificial Sequence
  the sequence is synthesized
gctcatagcagccaccttcat

SEQ ID NO: 66 - DNA, 20 bp, Artificial Sequence
  the sequence is synthesized
gcactgagatcttcctattg

SEQ ID NO: 67 - DNA, 21 bp, Artificial Sequence
  the sequence is synthesized
cgcgagctatagaagaatcac

SEQ ID NO: 68 - DNA, 19 bp, Artificial Sequence
  the sequence is synthesized
ttctgcttggggtcagcac

SEQ ID NO: 69 - DNA, 21 bp, Artificial Sequence
  the sequence is synthesized
gaactttcagaagggccaggt

SEQ ID NO: 70 - DNA, 22 bp, Artificial Sequence
  the sequence is synthesized
acatagatgtggtacagggagg

SEQ ID NO: 71 - DNA, 21 bp, Artificial Sequence
  the sequence is synthesized
gaactttcagaagggccaggt

SEQ ID NO: 72 - DNA, 20 bp, Artificial Sequence
  the sequence is synthesized
ttgcgctcaatctcctcctc

SEQ ID NO: 73 - DNA, 21 bp, Artificial Sequence
  the sequence is synthesized
ccaggcagagaatgctgagtt

SEQ ID NO: 74 - DNA, 22 bp, Artificial Sequence
  the sequence is synthesized
acatagatgtggtacagggagg

SEQ ID NO: 75 - DNA, 21 bp, Artificial Sequence
  the sequence is synthesized
gtcaggcagagggtgatagaa

SEQ ID NO: 76 - DNA, 21 bp, Artificial Sequence
  the sequence is synthesized
gtccttggtgtcatgggtcag

SEQ ID NO: 77 - DNA, 21 bp, Artificial Sequence
  the sequence is synthesized
agggtgatagaagaggccaag

SEQ ID NO: 78 - DNA, 20 bp, Artificial Sequence
  the sequence is synthesized
gctcctgcaactcctcaaag

SEQ ID NO: 79 - DNA, 20 bp, Artificial Sequence
  the sequence is synthesized
ggccagcaacaaagtgcaag

SEQ ID NO: 80 - DNA, 21 bp, Artificial Sequence
  the sequence is synthesized
gagtgtaaggacccatcggag

SEQ ID NO: 81 - DNA, 21 bp, Artificial Sequence
  the sequence is synthesized
caggtcattcagatgtagcgg

SEQ ID NO: 82 - DNA, 21 bp, Artificial Sequence
  the sequence is synthesized
actcttttggatgctctggtc

SEQ ID NO: 83 - DNA, 22 bp, Artificial Sequence
  the sequence is synthesized
cgagatgacttcgaaaagctga

SEQ ID NO: 84 - DNA, 22 bp, Artificial Sequence
  the sequence is synthesized
tcatgtattgctttgcgttgga

SEQ ID NO: 85 - DNA, 22 bp, Artificial Sequence
  the sequence is synthesized
gctgtgtggcaactttaacatc

SEQ ID NO: 86 - DNA, 22 bp, Artificial Sequence
  the sequence is synthesized
agttggcaaagtcataagggtc

SEQ ID NO: 87 - DNA, 21 bp, Artificial Sequence
  the sequence is synthesized
agtttcgccaaggctttcatt

SEQ ID NO: 88 - DNA, 21 bp, Artificial Sequence
  the sequence is synthesized
ctgacacctgagtgagacgag

SEQ ID NO: 89 - DNA, 22 bp, Artificial Sequence
  the sequence is synthesized
cctccagtttcccagcttctta

SEQ ID NO: 90 - DNA, 21 bp, Artificial Sequence
  the sequence is synthesized
ttccatactgcagcactgaca

SEQ ID NO: 91 - DNA, 19 bp, Artificial Sequence
  the sequence is synthesized
gacactggtctgcaccctt

SEQ ID NO: 92 - DNA, 19 bp, Artificial Sequence
  the sequence is synthesized
gactctccagcggctcaca

SEQ ID NO: 93 - DNA, 21 bp, Artificial Sequence
  the sequence is synthesized
ttgactctggacactggtctg

SEQ ID NO: 94 - DNA, 20 bp, Artificial Sequence
  the sequence is synthesized
aatccatgcttccgtggaca

SEQ ID NO: 95 - DNA, 20 bp, Artificial Sequence
  the sequence is synthesized
gctgcattgactctggacac

SEQ ID NO: 96 - DNA, 19 bp, Artificial Sequence
  the sequence is synthesized
gactctccagcggctcaca

SEQ ID NO: 97 - DNA, 21 bp, Artificial Sequence
  the sequence is synthesized
acgtggatgactgcatactgg

SEQ ID NO: 98 - DNA, 22 bp, Artificial Sequence
  the sequence is synthesized
gtcgtagttagggtagcagtgg

SEQ ID NO: 99 - DNA, 21 bp, Artificial Sequence
  the sequence is synthesized
acgtggatgactgcatactgg

SEQ ID NO: 100 - DNA, 21 bp, Artificial Sequence
  the sequence is synthesized
caggtcgtagttagggtagca

SEQ ID NO: 101 - DNA, 21 bp, Artificial Sequence
  the sequence is synthesized
ggacgtggatgactgcatact

SEQ ID NO: 102 - DNA, 20 bp, Artificial Sequence
  the sequence is synthesized
gaagccaccctgtgtgttga

SEQ ID NO: 103 - DNA, 20 bp, Artificial Sequence
  the sequence is synthesized
gggaactatgatgctgccaa

SEQ ID NO: 104 - DNA, 19 bp, Artificial Sequence
  the sequence is synthesized
ccgcaccatggccaaagaa

SEQ ID NO: 105 - DNA, 21 bp, Artificial Sequence
  the sequence is synthesized
ccaattacatcggctcagaca

SEQ ID NO: 106 - DNA, 22 bp, Artificial Sequence
  the sequence is synthesized
tctggatattctctctggcatc

SEQ ID NO: 107 - DNA, 20 bp, Artificial Sequence
  the sequence is synthesized
ggtcttgaccagcctctctc

SEQ ID NO: 108 - DNA, 21 bp, Artificial Sequence
  the sequence is synthesized
tccgactctttgggaaacaca

SEQ ID NO: 109 - DNA, 21 bp, Artificial Sequence
  the sequence is synthesized
ttcactgtgtgcctccacttc

SEQ ID NO: 110 - DNA, 21 bp, Artificial Sequence
  the sequence is synthesized
cgccttgcacttcatacttca

SEQ ID NO: 111 - DNA, 20 bp, Artificial Sequence
  the sequence is synthesized
agcctctcaaagccttcact

SEQ ID NO: 112 - DNA, 20 bp, Artificial Sequence
  the sequence is synthesized
tgaacacttcgccttgcact

SEQ ID NO: 113 - DNA, 19 bp, Artificial Sequence
  the sequence is synthesized
agatttggacctgcgagcg

SEQ ID NO: 114 - DNA, 20 bp, Artificial Sequence
  the sequence is synthesized
gagcggctgtctccacaagt

SEQ ID NO: 115 - DNA, 20 bp, Artificial Sequence
  the sequence is synthesized
ctttgctgctgcttgacaga

SEQ ID NO: 116 - DNA, 20 bp, Artificial Sequence
  the sequence is synthesized
gccttgttgttgttggcctt

SEQ ID NO: 117 - DNA, 20 bp, Artificial Sequence
  the sequence is synthesized
aactcaagccttaccgcaga

SEQ ID NO: 118 - DNA, 20 bp, Artificial Sequence
  the sequence is synthesized
tgcagcaggaagaagagtca

SEQ ID NO: 119 - DNA, 19 bp, Artificial Sequence
  the sequence is synthesized
tacactagccatccttact

SEQ ID NO: 120 - DNA, 19 bp, Artificial Sequence
  the sequence is synthesized
gaaggttttacaagactca

SEQ ID NO: 121 - DNA, 20 bp, Artificial Sequence
  the sequence is synthesized
gttacactagccatccttac

SEQ ID NO: 122 - DNA, 20 bp, Artificial Sequence
  the sequence is synthesized
gaaggttttacaagactcac

SEQ ID NO: 123 - DNA, 26 bp, Artificial Sequence
  the sequence is synthesized
acaggtacgttaatagttaatagcgt

SEQ ID NO: 124 - DNA, 22 bp, Artificial Sequence
  the sequence is synthesized
atattgcagcagtacgcacaca

SEQ ID NO: 125 - DNA, 22 bp, Artificial Sequence
  the sequence is synthesized
gtgaratggtcatgtgtggcgg

SEQ ID NO: 126 - DNA, 26 bp, Artificial Sequence
  the sequence is synthesized
caratgttaaasacactattagcata

SEQ ID NO: 127 - DNA, 21 bp, Artificial Sequence
  the sequence is synthesized
cgcgctactctctctttctgg

SEQ ID NO: 128 - DNA, 22 bp, Artificial Sequence
  the sequence is synthesized
agtcaacttcaatgtcggatgg

SEQ ID NO: 129 - DNA, 20 bp, Artificial Sequence
  the sequence is synthesized
catgtgtggcggttcactat

SEQ ID NO: 130 - DNA, 20 bp, Artificial Sequence
  the sequence is synthesized
tgcattaacattggccgtga

SEQ ID NO: 131 - DNA, 20 bp, Artificial Sequence
  the sequence is synthesized
ctacatgcaccagcaactgt

SEQ ID NO: 132 - DNA, 20 bp, Artificial Sequence
  the sequence is synthesized
cacctgtgcctgttaaacca

SEQ ID NO: 133 - DNA, 20 bp, Artificial Sequence
  the sequence is synthesized
caatgctgcaatcgtgctac

SEQ ID NO: 134 - DNA, 20 bp, Artificial Sequence
  the sequence is synthesized
gttgcgactacgtgatgagg

SEQ ID NO: 135 - DNA, 21 bp, Artificial Sequence
  the sequence is synthesized
gagatctctcaacgtgctcag

SEQ ID NO: 136 - DNA, 23 bp, Artificial Sequence
  the sequence is synthesized
cttggcataaaacaggttcagaa

SEQ ID NO: 137 - DNA, 21 bp, Artificial Sequence
  the sequence is synthesized
aaataccagtggcttaccgca

SEQ ID NO: 138 - DNA, 21 bp, Artificial Sequence
  the sequence is synthesized
gccaccagctcctttattacc

SEQ ID NO: 139 - DNA, 20 bp, Artificial Sequence
  the sequence is synthesized
ggtagcagaactcgaaggca

SEQ ID NO: 140 - DNA, 21 bp, Artificial Sequence
  the sequence is synthesized
atgagggacaaggacaccaag

SEQ ID NO: 141 - DNA, 23 bp, Artificial Sequence
  the sequence is synthesized
tccctgacttaaatggtgatgtg

SEQ ID NO: 142 - DNA, 23 bp, Artificial Sequence
  the sequence is synthesized
tcttaaaagagggtgtgtagtgt

SEQ ID NO: 143 - DNA, 21 bp, Artificial Sequence
  the sequence is synthesized
gcctgctgaaaatgactgaat

SEQ ID NO: 144 - DNA, 22 bp, Artificial Sequence
  the sequence is synthesized
attagctgtatcgtcaaggcac